(19) United States
(12) Patent Application Publication
(10) Pub. No.: US 2014/0004578 A1
(43) Pub. Date: Jan. 2, 2014

(54) MICROBES, METHODS, AND DEVICES FOR REDOX-IMBALANCED METABOLISM

(71) Applicants: Daniel R. Bond, St. Paul, MN (US); Jeffrey A. Gralnick, St. Paul, MN (US); Daniel E. Ross, Cayce, SC (US); Jeffrey M. Flynn, Madison, WI (US)

(72) Inventors: Daniel R. Bond, St. Paul, MN (US); Jeffrey A. Gralnick, St. Paul, MN (US); Daniel E. Ross, Cayce, SC (US); Jeffrey M. Flynn, Madison, WI (US)

(73) Assignee: REGENTS OF THE UNIVERSITY OF MINNESOTA

(21) Appl. No.: 13/822,837

(22) PCT Filed: Sep. 13, 2011
(86) PCT No.: PCT/US11/51396

§ 371 Date: Sep. 17, 2013

Related U.S. Application Data
(60) Provisional application No. 61/382,139, filed on Sep. 13, 2010.

(51) Int. Cl. C12N 13/00

(52) U.S. Cl. CPC C12N 13/00
USPC 435/134; 435/170; 435/289.1; 435/252.3; 435/252.33; 435/252.34; 435/136; 435/145; 435/166

(57) ABSTRACT

The invention generally relates to methods, devices, and microbes involved in performing redox imbalanced fermentations. In one aspect, the invention provides a device that generally includes an electrode and at least one microbe in electron communication with the electrode. The microbe generally can exhibit increased activity of at least one enzyme involved in converting a substrate to a redox imbalanced product. In another aspect, the invention provides a method for performing redox imbalanced fermentation. Generally, the method includes providing a substrate to a microbe under conditions effective for the microbe to metabolize the substrate to a redox imbalanced product. At least one microbe may be in contact with an electrode. In some cases, metabolic conversion of the substrate to the redox imbalanced product can include transferring electrons between the electrode and the microbe. In other cases, metabolic conversion of the substrate to the redox imbalanced product exhibits a carbon flux from organic substrate to organic product of at least 80%. In another aspect, the invention provides a genetically modified Shewanella oneidensis microbe.

### CROSS-REFERENCE TO RELATED APPLICATION

This application claims priority to U.S. Provisional Patent Application Ser. No. 61/382,139, filed Sep. 13, 2010.

### BACKGROUND

The combination of metabolic engineering tools and a need to efficiently convert feedstocks to products or fuels has resulted in the engineering of many strains of microbes that are able to catalyze useful fermentations. Many challenges remain, however, to increase the efficiency of such fermentation processes. Many challenges also remain with respect to engineering the systems to reduce the number and amount of undesirable metabolic products.

### SUMMARY OF THE INVENTION

The invention generally relates to methods, devices, and microbes involved in performing redox imbalanced fermentations.

In one aspect, the invention provides a device that generally includes an electrode and at least one microbe in electron communication with the electrode. The microbe generally exhibits increased activity, compared to a wild-type control, of at least one enzyme involved in redox imbalanced metabolism. In some cases, the microbe can be an electron flux microbe genetically modified to exhibit increased activity of at least one enzyme that catalyzes a metabolic step converting a substrate to a redox-imbalanced pathway product. In other cases, the microbe can be genetically modified to exhibit increased activity of at least one enzyme that catalyzes a metabolic step converting a substrate to a redox-imbalanced pathway product.

In another aspect, the invention provides a method for performing a redox imbalanced fermentation. Generally, the method includes providing a substrate to a microbe under conditions effective for the microbe to metabolize the substrate to a redox imbalanced product. At least one microbe may be in contact with an electrode. Also, metabolic conversion of the substrate to the redox imbalanced product can include transferring electrons between the electrode and the microbe.

In another aspect, the invention provides an alternative method for performing a redox imbalanced fermentation. Generally, the method includes providing a substrate to a microbe under conditions effective for the microbe to metabolize the substrate to a redox imbalanced product. In this case, at least one microbe is in contact with an electrode and metabolic conversion of the substrate to the redox imbalanced product exhibits a carbon flux from organic substrate to organic product of at least 80%.

In another aspect, the invention provides a genetically modified Shewanella oneidensis microbe. Generally, the microbe is genetically modified to exhibit increased activity, compared to a wild-type control, of at least one enzyme that catalyzes a metabolic step converting a substrate to a redox-imbalanced pathway product. In one embodiment, the microbe can include at least one heterologous coding sequence that encodes an enzyme involved in converting glycerol to dihydroxyacetone phosphate and at least one heterologous coding sequence that encodes an enzyme involved in converting pyruvate to ethanol.

In another aspect, the invention provides a genetically modified Escherichia coli microbe. Generally, the microbe is genetically modified to exhibit increased activity, compared to a wild-type control, of at least one enzyme that catalyzes electron flux across the microbe's outer membrane. In one embodiment, the microbe can include at least one heterologous coding sequence that encodes at least one of MtrA, MtrB, MtrC.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Metabolic modules added to S. oneidensis to enable electrode-dependent conversion of glycerol to ethanol. Glycerol utilization module from E. coli (top box) and ethanol production module from Z. mobilis (lower box) were combined with native metabolic pathways (middle box) for stoichiometric non-redox balanced conversion of glycerol to ethanol. The genes from E. coli and Z. mobilis engineered into S. oneidensis are represented by the proteins they encode: GlpF, glycerol transporter; GlpK, glycerol kinase; GlpD, glycerol-3-phosphate dehydrogenase; Pdc, pyruvate decarboxylase; and AdhB, alcohol dehydrogenase. Pta, phosphotransacetylase is shown from native metabolism. Electrons not redox balanced within the cell are subsequently transferred through the Mtr pathway to the electrode. Dots represent flavins secreted naturally from cells to accelerate extracellular electron transfer.

FIG. 2. Glycerol utilization and ethanol production in engineered S. oneidensis strains. (A) Plasmid map of pGUT2PET. (B) Aerobic growth of S. oneidensis with pGUT2PET (▴, σ), S. oneidensis with empty vector (▪, ν), and E. coli K12 (, λ) on glycerol. Anaerobic resting cell fumarate batch reactions with (C) wild-type with pGUT2PET and (D) Δpta with pGUT2PET. In panels C and D, glycerol (, λ), ethanol (▴, σ) and acetate (▪, ν) concentrations were determined by analyzing culture supernatants. Error bars represent standard deviation of at least three independent experiments.

FIG. 3. Bioelectric conversion of glycerol to ethanol. (A) Three-electrode bioreactor setup for electrode-dependent conversion of glycerol to ethanol. (B) Glycerol consumption and ethanol production in wild type strain pGUT2PET and (C) Δpta strain pGUT2PET on a graphite electrode. Glycerol (, λ) and acetate (▪, ν) concentrations were determined by HPLC. Error bars represent standard deviation of at least three independent experiments. Ethanol concentrations (▴, σ) were predicted based on average reaction stoichiometries for glycerol consumption and coloumbic yield from three independent experiments. Abiotic or non-poised electrode control maintained a constant glycerol concentration (□).

FIG. 4. Microbial electrochemical conversion of glycerol to ethanol. (A, B) Representative chronoamperometry (CA) of current produced from the conversion of glycerol to ethanol in three-electrode bioreactors (n=3) inoculated with (A) wild type strain pGUT2PET and (B) Δpta strain pGUT2PET. At time zero, ˜1.0 O.D. of cells was added to the reactor. (C, D) Determining the coulombic efficiency of engineered pathways. Representative data of real-time, continuously measured charge (λ) and total calculated charge (ν) from stoichiometric conversion of glycerol to ethanol for (C) wild type strain pGUT2PET and (D) Δpta strain pGUT2PET. The measured charge (λ) was determined from current data in panel A or B and continuously measured during the experiment. The calculated charge (ν) is based upon the stoichiometry of the reaction mechanism and was determined when samples from panel A or B were extracted for HPLC analysis.

FIG. 5. Electrode-dependent fumarate reduction in S. oneidensis MR-1. Representative chronoamperometry (CA) of S. oneidensis MR-1 thin films on graphite electrodes. Electrodes were poised at ˜0.36 V versus SHE and after 0.5 hours 50 mM fumarate was added to stirred bioreactors.

FIG. 6. Single turnover and catalytic voltammetry of WT and ΔfccA thin films attached to electrodes. Representative cyclic voltammograms (1 mV/s) of fumarate responses of S. oneidensis MR-1 (no addition, long arrow; 50 mM fumarate, short arrow) and ΔfccA (no addition, dark gray trace; 50 mM fumarate, light gray trace) after 16 hr of attachment to electrodes poised at +0.24 V versus SHE. The redox peak centered at +0.2 V versus SHE is indicative of a redox active species in close proximity to the electrode surface, i.e. c-type cytochromes exposed on the outer membrane.

FIG. 7. Components of the Mtr pathway are required for inward electron flux. Representative chronoamperometry of Mtr mutant thin films on electrodes poised at −0.36 V versus SHE. Current was constantly monitored and at 0.6 hours, 50 mM fumarate was added to stirred bioreactors.

FIG. 8. Rate of inward electron flux normalized to total electrode-attached protein, showing effect of riboflavin. Maximum current responses after fumarate addition (open bars) were normalized to attached protein values to obtain a specific rate of electron transfer (μA/μg protein; n=3, ±standard deviation). Patterned bars represent maximum current values after addition of 1 μM riboflavin.

FIG. 9. Riboflavin causes a shift in the potential required for reductive electron flow into ΔcymA. Representative cyclic voltammograms (1 mV/s) of: S. oneidensis MR-1 with no additions (dotted lines), 50 mM fumarate (dashed lines), and 50 mM fumarate+1 μM riboflavin (solid lines) for (A) wild type thin films and (B) ΔcymA thin films. (C) Derivative plots showing midpoint potentials for WT (black traces) and ΔcymA (gray traces). The midpoint potential for the menC mutant was similar to ΔcymA.

FIG. 10. A model for reversible electron transfer through the Mtr respiratory pathway in S. oneidensis MR-1. (A) Electrons generated at the electrode surface are transferred to MtrC. MtrC then transfers electrons to MtrA by interacting through MtrB. From MtrA electrons are passed to CymA and through themenaquinone pool to a second CymA interacting with FccA. Approximately 85% of inward electron flux is dependent on flow through the menaquinone pool, while approximately 15% relies on transfer from MtrA to FccA. Multi-heme cytochromes include BtrC, MtrA, CymA, and FccA; non-heme proteins include MtrB. (B) Redox potential windows for components involved in electrode-dependent fumarate reduction in S. oneidensis. Lines at approximately −250 mV, −220 mV, and −175 mV represent midpoint potentials for specific hemes within CymA or FccA. The line at approximately −150 mV represents the midpoint potential of the FAD cofactor of FccA.

FIG. 11. Engineering a nearly cytochrome-less Shewanella. A) Schematic of Shewanella cytochrome network. B) Plasmid map of pmtrCABcymA. C) Herne stained SDS-PAGE and immunoblot of MtrB in whole cell extracts of S. oneidensis MR-1, S. oneidensis ΔOMC/ΔPEC/ΔcymA, S. oneidensis ΔOMC/ΔPEC/ΔcymA+pCABcymA, E. coli BL21(DE3), and E. coli BL21(DE3)+pmtrCABcymA.

FIG. 12. Characterization of S. oneidensis and E. coli strains. (A-C) Fe(III)-citrate and Fe(III)-oxide reduction kinetics for WT MR-1, ΔOMC/ΔPEC/ΔcymA, and ΔOMC/ΔPEC/ΔcymA+pmtrCABcymA. (D) Fe(III)-oxide reduction kinetics for E. coli BL21(DE3) and E. coli BL21(DE3) pEC86+pmtrCABcymA. Specific activity of S. oneidensis (E) and E. coli (F) strains towards amorphous Fe(III)-oxide expressed as nmol Fe(II) formed/min/mg protein in the presence and absence of added riboflavin (1 μM).

FIG. 13. Growth and respiration-linked electrode reduction. Representative chronoamperometry (CA) traces of S. oneidensis MR-1, S. oneidensis ΔOMC/ΔPEC/ΔcymA, and S. oneidensis ΔOMC/ΔPEC/ΔcymA+pmtrCABcymA. B) Representative growth, medium exchange, and mediator addition of E. coli BL21(DE3) pEC86+pmtrCABcymA biofilms.

FIG. 14. (A) Current densities of S. oneidensis (1), ΔOMC/ΔPEC/ΔcymA (2), ΔOMC/ΔPEC/ΔcymA+pmtrCABcymA (3), E. coli BL21(DE3) (4) and E. coli BL21(DE3) pEC86+pmtrCABcymA (5) before (−) and after (+) replacement with mediator-free medium. (B) Current normalized to electrode-attached biomass (μA/μg).

FIG. 15. (A) Cyclic voltammetry (CV) of S. oneidensis and E. coli strains in the presence of electron donor (lactate) at maximal current densities and (B) after replacement with mediator-free medium, showing the relationship between current and voltage.

FIG. 16. Electron flux into E. coli: (A) Representative chronoamperometry (CA) data of a BL21(DE3) pEC86 pmtrCABcymA biofilm grown on a poised electrode (+0.44 V vs SHE), washed free of planktonic cells, and equilibrated at a negative electrode potential (−0.36 V vs SHE). After poising for 90 minutes, 50 mM fumarate was added. (B) Representative cyclic voltammetry data of electrode-attached E. coli before and after the addition of fumarate. The fumarate-dependent anodic wave in the low potential window suggests that electrons supplied by the electrode are reaching the fumarate reductase localized to the cytoplasmic membrane in E. coli. E. coli strain BL21(DE3) was transformed with pEC86 (cytochrome c maturation genes) and pmtrCABcymA (plasmid containing components of the Mtr respiratory pathway from Shewanella oneidensis MR-1. BL21(DE3) pEC86 pmtrCABcymA was grown at 30° C. overnight in LB supplemented with 50 μg/mL kanamycin and 34 μg/mL chloramphenicol. This cell suspension was centrifuged, washed twice in Shewanella Basal Medium (SBM) and resuspended in anaerobic SBM supplemented with vitamins, minerals, 1 μM riboflavin and 30 mM lactate. Ten milliliters of the final cell suspension (O.D.₆₀₀ ˜1) was used to inoculate an anaerobic 3-electrode bioreactor with a working electrode poised at +0.44 V vs. SHE. Once a stable plateau current was reached, the medium containing planktonic cells was removed, and replaced with fresh anaerobic SBM (with vitamins, minerals, riboflavin and lactate). After washing, the electrode potential was changed to −0.36 V vs SHE and anaerobic fumarate was added to a final concentration of 50 mM once a stable current was obtained.

FIG. 17. A plasmid containing glpF and gldA from Escherichia coli was mated into S. oneidensis MR-1. Single colonies were picked from plates and inoculated into 3 mL LB or LB supplemented with 50 μg/mL kanamycin and grown to an OD₆₀₀ of 0.6. This cell suspension was washed twice in SBM and diluted to an OD₆₀₀ of ˜0.05 in SBM containing vitamins, minerals, casamino acids and 50 mM glycerol and grown aerobically.

### DETAILED DESCRIPTION OF ILLUSTRATIVE EMBODIMENTS

All reactions catalyzed by whole cells or enzymes must achieve redox balance. In rare cases, conversion can be achieved via perfectly redox balanced fermentations, allowing all electron equivalents to be recovered in a single product (e.g., sugar fermentation to ethanol). In most biotransformation, however, organisms must produce a mixture of side products such as acids, gasses, and/or alcohols in order to achieve redox balance. No amount of enzyme or strain engineering can overcome this fundamental requirement that reactions be redox balanced.

The present invention involves using microbes in contact with electrodes in order to provide a non-chemical electron source/electron sink so that the microbe can achieve redox balance without being forced to produce undesired side products. As a result, redox imbalanced fermentations can produce desired products at higher yield and with less contamination by side products than is otherwise possible.

The combination of metabolic engineering tools and a need to efficiently convert feedstocks to products or fuels has resulted in the engineering of many strains of microbes that are able to catalyze useful fermentations. The need to achieve redox balance, however, limits the range of possible bioconversions. Moreover, the synthesis of mixtures of end products increases bioseparation costs. One possible solution to these challenges is to exploit the ability of certain bacteria to transfer electrons to electrodes. Some microorganisms have previously been shown to utilize exogenous redox mediators (e.g. thionine, neutral red, or ferricyanide) to balance redox reactions (Emde et al., 1989 Appl. Microbiol. Biotechnol. 32(2):170-175; Park and Zeikus, 1999 J. Bacteriol. 181(8):2403-10). In addition, certain bacteria can natively transfer electrons directly to electrodes (Logan, 2009 Nat. Rev. Microbiol. 7(5):375-381; Lovley, 2008 Curr. Opin. Biotechnol. 19(6):564-571), a process called extracellular electron transfer.

Extracellular electron transfer allows bacteria to utilize an electrode as an external sink for electrons, reducing the production of undesired side products merely to achieve redox balance. Extracellular electron transfer can thereby facilitate stoichiometric conversion of substrate to product while simultaneously generating electrical current.

Cellular metabolism is a series of tightly linked oxidations and reductions that must be balanced. Recycling of intracellular electron carriers during fermentation often requires substrate conversion to undesired products, while respiration demands constant addition of electron acceptors. The use of electrode-based electron acceptors to balance biotransformations would overcome these constraints.

We have engineered the metal reducing bacterium Shewanella oneidensis to stoichiometrically convert a substrate such as, for example, glycerol to a redox imbalanced product such as, for example, ethanol. In the representative exemplary embodiment in which glycerol is converted to ethanol, biotransformation will not occur unless two electrons are removed via an external reaction. We achieve the removal of the two electrons by electrode reduction. Multiple modules were combined into a single plasmid to alter S. oneidensis metabolism. One module, referred to herein as a glycerol module, includes glpF, glpK, glpD and tpiA from Escherichia coli. A second module, referred to herein as an ethanol module, includes pdc and adh from Zymomonas mobilis. A further increase in product yields was accomplished through knockout of pta, which encodes phosphate acetyltransferase. The pta knockout shifts flux towards ethanol and away from acetate production. We converted glycerol to ethanol by using an electrode to balance the reaction. Electrode-linked glycerol-to-ethanol conversion rates were on par with volumetric conversion rates observed in engineered E. coli. Linking microbial biocatalysis to current production can eliminate redox constraints by shifting other unbalanced reactions to yield pure products and serve as a new platform for next generation bioproduction strategies.

As noted above, most biotransformations result in organisms producing a mixture of acids, gasses, and/or alcohols, and no amount of enzyme or strain engineering can overcome this fundamental requirement. Stoichiometric conversion of glycerol, a waste product from biodiesel transesterfication, into ethanol without a concomitant production of side products represents such an otherwise impossible fermentation because glycerol exists in a reduced redox state compared to ethanol. The unbalanced conversion of glycerol to ethanol has, until now, been viewed as having only two solutions: fermenting glycerol to ethanol plus co-products, requiring separation of ethanol from the co-products, or “burning off” excess electrons via careful introduction of oxygen. In contrast, we have developed a third strategy that achieves redox-balanced conversion of glycerol to ethanol by using bacteria directly interfaced to electrodes.

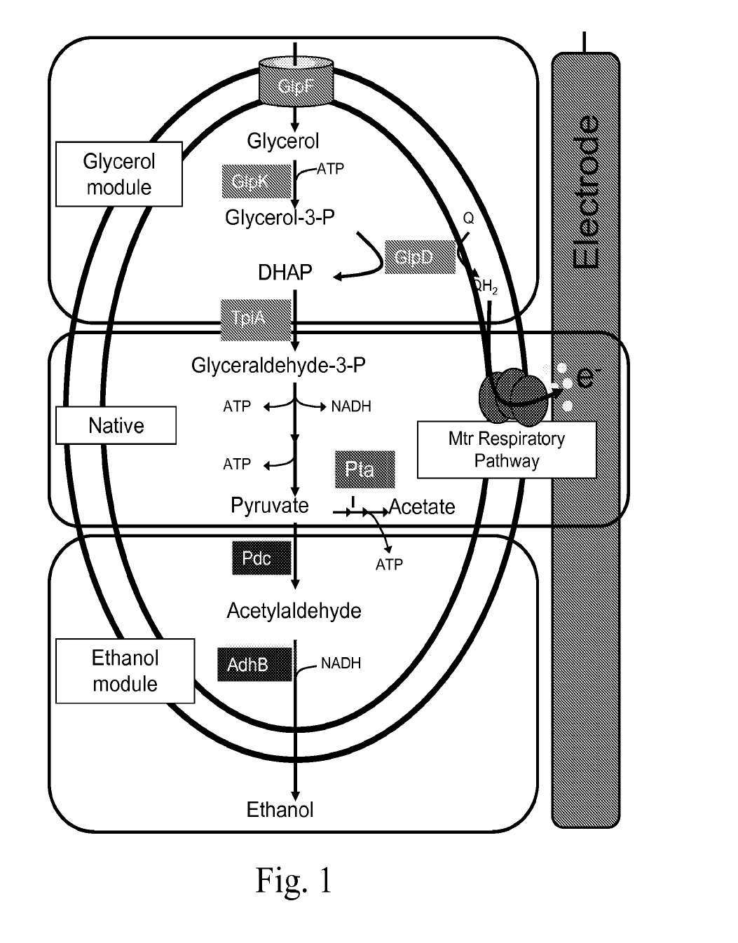

In one aspect, the invention involves a genetically modified microorganism capable of transferring metabolic electrons to an extracellular electron sink such as, for example, an electrode. In one exemplary embodiment, we used the facultative anaerobe Shewanella oneidensis to produce ethanol from glycerol without co-producing redox-balancing end products such as, for example, formate or 1,2 propanediol. S. oneidensis possesses the ability to respire electrons to insoluble substrates such as electrodes (Hartshorne et al., 2009 Proc. Natl. Acad. Sci. USA. 106(52):22169-22174; Hau and Gralnick, 2007 Ann. Rev. Microbiol. 61:237-58), a diverse metabolism, and a sequenced genome (Heidelberg et al., 2002 Nat. Biotechnol. 20(11):1118-1123) amenable to genetic manipulations. Unlike common industrial hosts such as Escherichia coli, S. oneidensis is naturally equipped for electron transfer to electrode surfaces. It localizes a well-characterized protein complex (MtrCAB) to span the periplasm and outer membrane, in addition to an inner membrane tetraheme protein (CymA) capable of transferring electrons from the respiratory quinone pool to MtrCAB (Hartshorne et al., 2009 Proc. Natl. Acad. Sci. USA. 106(52):22169-22174; Ross et al., 2007 Appl. Environ. Microbiol. 73:5797-5808; Shi et al., 2007 Mol. Microbiol. 65:12-20). The MtrCAB complex can directly reduce insoluble substrates (Hartshorne et al., 2009 Proc. Natl. Acad. Sci. USA. 106(52):22169-22174; Ross et al., 2009 Appl. Environ. Microbiol. 75(16):5218-5226) though its activity is dramatically increased in the presence of low concentrations of flavins (Ross et al., 2009 Appl. Environ. Microbiol. 75(16):5218-5226; Baron et al., 2009 J. Biol. Chem. 284(42):28865-28873). Flavins have recently been shown to be secreted by Shewanella (Marsili et al., 2008 Proc. Natl. Acad. Sci. U.S.A. 105(10):3968-3973; von Canstein et at, 2008 Appl. Environ. Microbiol. 74(3):615-623) and are themselves reduced by the Mtr pathway (Coursolle et al., 2010 J. Bacteriol. 192(2):467-474). These electron transfer capabilities are the foundation for a demonstration to enable an otherwise unbalanced fermentation utilizing bacteria interfaced with an electrode. Two genetic modules were engineered into S. oneidensis to allow for glycerol utilization and ethanol production in a manner that would feed directly into this electrode respiration machinery (FIG. 1).

In another aspect, the invention relates to methods of performing redox imbalanced metabolic reactions. Here, we report the electrode-dependent conversion of glycerol to ethanol in an engineered strain of S. oneidensis, and while our work focuses on solving this specific unbalanced redox reaction, our strategy can be broadly applied to any reaction where the substrate is more reduced than the desired product.

In addition, depending upon the particular modules engineered into the microbe, our strategy may be applied to any reaction where the substrate is more oxidized than the desired product by reversing the direction of electron flow—i.e., providing electron flow into microbe through the electrode. Thus, while the following description may refer to the redox reduction of glycerol to ethanol and certain benefits, observations, and other effects of such a pathway, it is readily apparent that corresponding benefits, observations, and other effects can be exploited by reversing the direction of electron flow. Consequently, the specific benefits, observations, and other effects expressly described below should not be construed to be generally limiting.

### DEFINITIONS

“Carbon flux” refers to the rate at which carbon atoms are exchanged between pools. In the context of the methods described herein, carbon flux can refer to the rate at which carbon atoms in a metabolic substrate are converted to carbon atoms in a metabolic product.

“Coding sequence” refers to any nucleotide sequence that may be transcribed and the transcription product translated to produce an amino acid sequence possessing a biological function such as, for example, an enzymatic function.

“Electrode-dependent metabolism” refers to metabolism (e.g., redox imbalanced fermentations) that includes the transfer of electrons between one or more microbes performing the metabolism and an electrode. The term does not imply any particular direction of electron transfer between microbe and electrode and may, indeed, involve either redox reduction or oxidation. Also, the term does not imply direct physical contact between the microbe and the electrode—i.e., the transfer of electrons can include an intermediary electron that shuttles electrons between the microbe and the electrode.

“Electron communication” refers to two materials between which electrons may be passed, either directly or indirectly. Indirect electron communication may be accomplished through an intermediate such as, for example, an electron shuttle.

“Redox imbalanced” refers generally to metabolism in which the redox state of the substrate and the redox state of the product differ. “Redox imbalanced” may refer to a particular metabolic pathway in which the redox state of the substrate and the redox state of the product differ so that balancing a redox imbalanced pathway requires either net input or net withdrawal of electrons. “Redox imbalanced” also may refer to a particular substrate and/or particular product of a specified metabolic pathway to indicate that the redox state of the substrate and the redox state of the product differ. For example, the metabolic conversion of glycerol to ethanol may be characterized as “redox imbalanced” because it involves the net withdrawal of electrons. Thus, ethanol may be characterized as a “redox imbalanced” product of glycerol metabolism.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Engineering of S. oneidensis for Glycerol Uptake and Utilization.

No Shewanella isolates tested to date have been shown to utilize glycerol as a sole carbon and energy source (Venkateswaran et al., 1999 Int. J. Syst. Bacteriol. 49:705-724), and growth experiments confirmed this inability in S. oneidensis strain MR-1. To create a respiratory pathway linked to quinone reduction, in contrast to fermentative pathways developed for E. coli (Gonzalez et al., 2008 Metabol. Eng. 10(5):234-245), three coding sequences were predicted to be required: glpF, glpK, and glpD, which encode a glycerol facilitator, glycerol kinase and a membrane-bound quinone-linked glycerol-3-phosphate dehydrogenase, respectively (da Silva et al., 2009 Biotechnol. Adv. 27(1):30-39). When these coding sequences were introduced under control of a lac promoter, glycerol kinase and glycerol dehydrogenase activities were detected in whole cell lysates, but the Shewanella did not grow on or utilize glycerol under any of the conditions tested. Only after introduction of tpiA, which encodes a triosephosphate isomerase responsible for isomerization of dihydroxyacetone phosphate (DHAP) and 3-phosphoglyceraldehyde, were glycerol consumption and cell growth observed. A requirement for increased TpiA activity was consistent with the known allosteric inhibition of GlpD by DHAP (18), and use of Entner-Duodoroff glycolysis by S. oneidensis (Scott and Nealson, 1994 J. Bacteriol. 176(11):3408-3411), which would only require low TpiA activity for gluconeogenic flux during utilization of its preferred substrate (lactate) as a carbon source.

Introducing these four constitutively expressed coding sequences (glpF, glpK, glpD, and tpiA) from E. coli (glycerol module, FIG. 1) enabled S. oneidensis to utilize glycerol as a sole carbon and energy source. The glpD coding sequence encodes a flavoenzyme characterized as the aerobic dehydrogenase of E. coli (Austin and Larson, 1991 J. Bacteriol. 173(1):101-107), while another enzyme complex (encoded by glpABC) is utilized for anaerobic utilization of glycerol by E. coli. However, S. oneidensis was able to utilize glycerol both aerobically and anaerobically with the GlpD dehydrogenase, suggesting it would be compatible with menaquinone-dependent transport of electrons via CymA to electrodes in S. oneidensis (Shi et al., 2007 Mol. Microbiol. 65:12-20). Moreover, the selective pressure presented by the carbon source was sufficient to maintain the plasmid in the S. oneidensis without further reliance on, for example, providing antibiotic resistance.

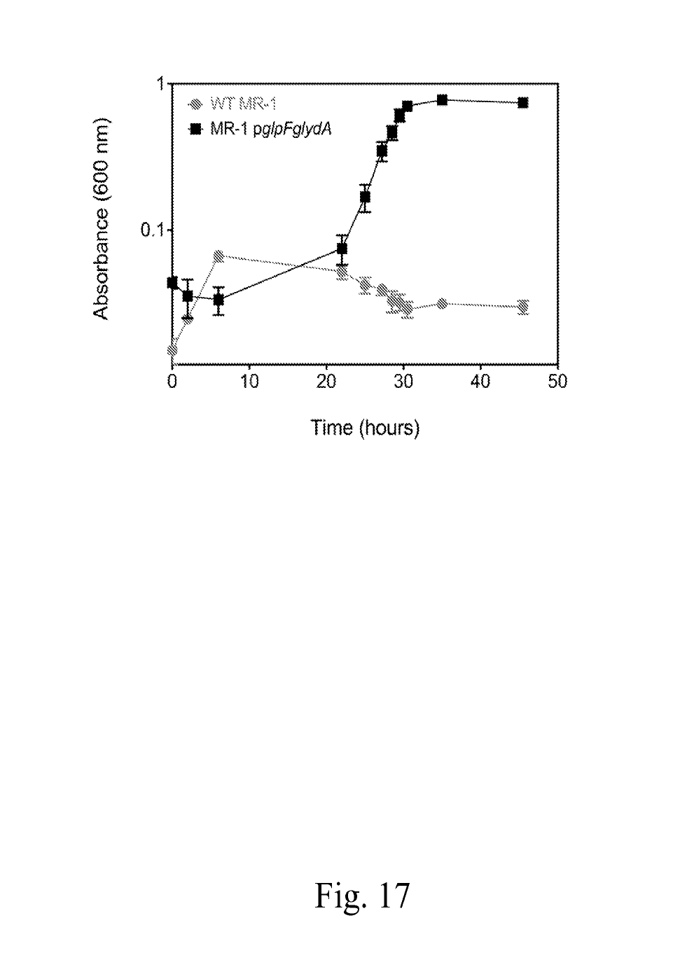

In another embodiment, a plasmid containing glpK and gldA from E. coli were introduced into S. oneidensis. This alternative glycerol module also allowed S. oneidensis to grow on glycerol as a sole carbon and energy source (FIG. 17), thus demonstrating the generality of our microbe engineering strategy.

Engineering of S. oneidensis for Ethanol Production.

Ethanol production has yet to be observed in wild-type S. oneidensis despite numerous putative alcohol dehydrogenases annotated in its genome. Attempts to increase pyruvate levels in S. oneidensis by knocking out acetate-producing pathways and/or overexpressing typical acetyl-CoA dependent alcohol dehydrogenases from E. coli have proved unsuccessful. Even in organisms with functional alcohol dehydrogenases such as, for example, E. coli, it is well established that expression of two coding sequences from Zymomonas mobilis (pdc, encoding pyruvate decarboxylase and adh, encoding alcohol dehydrogenase) can significantly increase ethanol production rates (Conway et al., 1987 J. Bacteriol. 169(6):2591-2597; Ingram et al., 1987 Appl. Environ. Microbiol. 53(10):2420-2425). The effectiveness of this route has been attributed to the high affinity of Pdc for pyruvate (Brau and Sahm, 1986 Arch. Microbiol. 146:105-110; Bringer-Meyer et al., 1986 Arch. Microbiol. 146(2):105-110). When pdc and adh were cloned and expressed constitutively in S. oneidensis (ethanol module, FIG. 1), pyruvate decarboxylase activity and alcohol dehydrogenase activity were observed in crude extracts and ethanol production from lactate was observed in cell suspensions.

Combination of Glycerol Utilization and Ethanol Production Modules for Conversion of Glycerol into Ethanol.

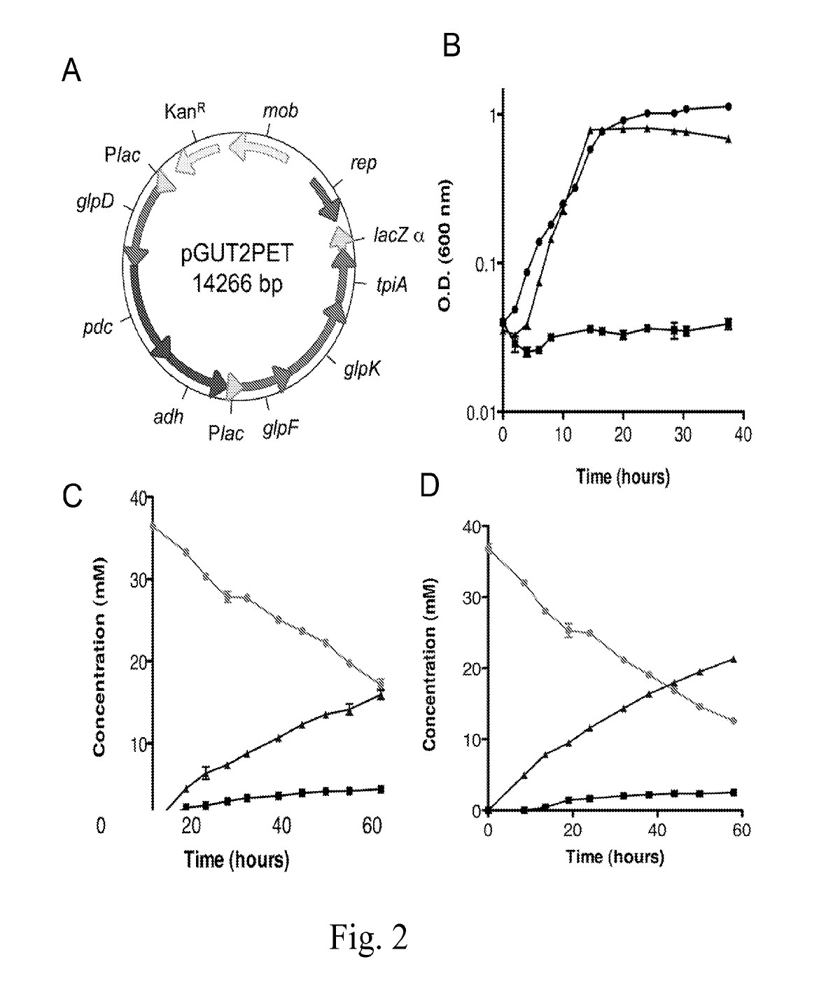

Glycerol utilization and ethanol production modules were incorporated into a single plasmid that encoded a total of six coding sequences in two operons each driven with a lac promoter. However, because Shewanella obtains more energy via substrate level phosphorylation by directing flux to acetate excretion compared to ethanol production (FIG. 1, (Hunt et al., 2010 J Bacteriol. 192(13):3345-51)), recombination events between lac promoter regions eliminating pdc and adh were common in initial constructs. However, by altering the coding sequence order on the plasmid, cloning pdc and adh directly downstream of glpD, the selective pressure to maintain glycerol metabolism also maintained ethanol production even in Shewanella strains with native recombination mechanisms, resulting in the construct pGUT2PET (FIG. 2A). A series of ten transfers of aerobic growth with glycerol yielded an adapted isolate of S. oneidensis with pGUT2PET that grew at rates comparable to wild-type E. coli provided with glycerol (FIG. 2B).

The pathway introduced by pGUT2PET was designed to enable conversion of glycerol to ethanol only when a mechanism was available for respiratory removal of two electrons. To demonstrate anaerobic glycerol conversion to ethanol and to examine its requirement for an external electron sink, batch cultures with or without the soluble electron acceptor fumarate were used to evaluate metabolic flux. When resting cells were incubated in sealed anaerobic tubes in the absence of an electron acceptor, no fermentative conversion of glycerol to any end product was detected. However, when fumarate was available, cells consumed 19.3±0.6 mM glycerol and produced 15.9±1.0 mM ethanol and 4.4±0.2 mM acetate as sole end products (FIG. 2C, Table 3). Recovery of electrons via fumarate reduction was stoichiometric, demonstrating that expression of these six coding sequences was sufficient to enable conversion of glycerol to ethanol, with 80% of the carbon flux directed to ethanol and 20% to acetate.

Generally, the methods described herein may result in carbon flux from substrate to product of at least 50%, at least 55%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

In E. coli, ethanol yield may be affected by competing metabolic pathways that produce a variety of contaminating products such as, for example, succinate, formate, lactate, 1,2 propanediol and/or acetate. In contrast, Shewanella only excretes acetate as a contaminating product (FIG. 2C). Therefore, to enhance flux to ethanol, an in-frame deletion of the pta coding sequence (encoding phosphate acetyltransferase), which is required for conversion of acetyl-CoA to acetyl-phosphate, was constructed in S. oneidensis (Castaño-Cerezo et al., 2009 Microb. Cell Fact. 8(54); Contiero et al., 2000 J. Ind. Microbiol. Biotechnol. 24(6):421-430; Kakuda et al., 1994 J. Biochem. 116(4):916-922). Transformation of the pGUT2PET plasmid into the Δpta background resulted in a 43% decrease in acetate production and a 33% increase in ethanol production compared to wild-type (FIG. 2D, Table 3).

Balancing Engineered Metabolic Pathways with Electrodes.

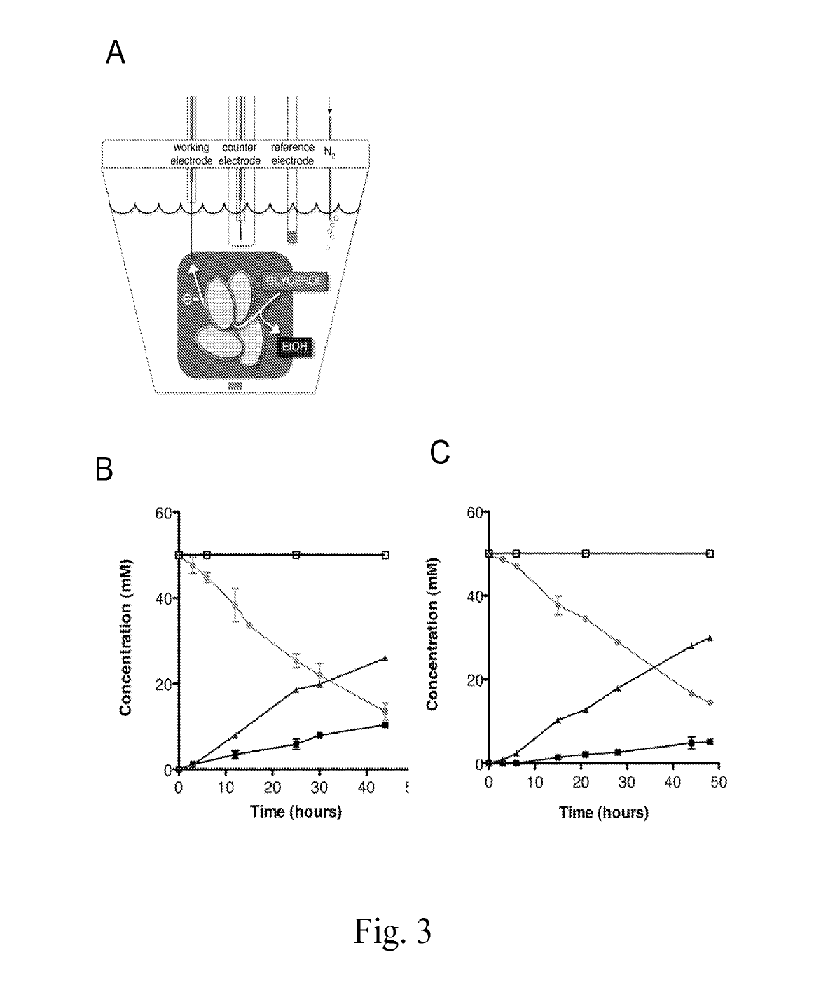

Our ultimate goal was use electrode-linked redox balancing to reduce as much of the production of side products as possible. As S. oneidensis pGUT2PET strains were unable to convert glycerol to ethanol in the absence of electron acceptors, electrode-dependent conversion of glycerol to ethanol could be tested in a bioreactor containing an electrode poised at an oxidizing potential (FIG. 3A). In these strictly anaerobic reactors, a carbon fiber electrode was poised with a potentiostat to act as a constant electron acceptor at +0.4 V versus a standard hydrogen electrode (SHE), allowing electrons to be collected and returned to the reactor via a Pt counter electrode.

In electrode bioreactors, no glycerol consumption was observed by wild-type or Δpta strains containing pGUT2PET when the electrode was disconnected. Fermentation was not stimulated by vigorous flushing with oxygen-free nitrogen or argon, which will enable fermentation of glycerol by E. coli (Yazdani and Gonzalez, 2008. Metabol. Eng. 10(6):340-351), and no glycerol was consumed in sterile reactors when electrodes were held at an oxidizing potential (FIG. 3B, 3C).

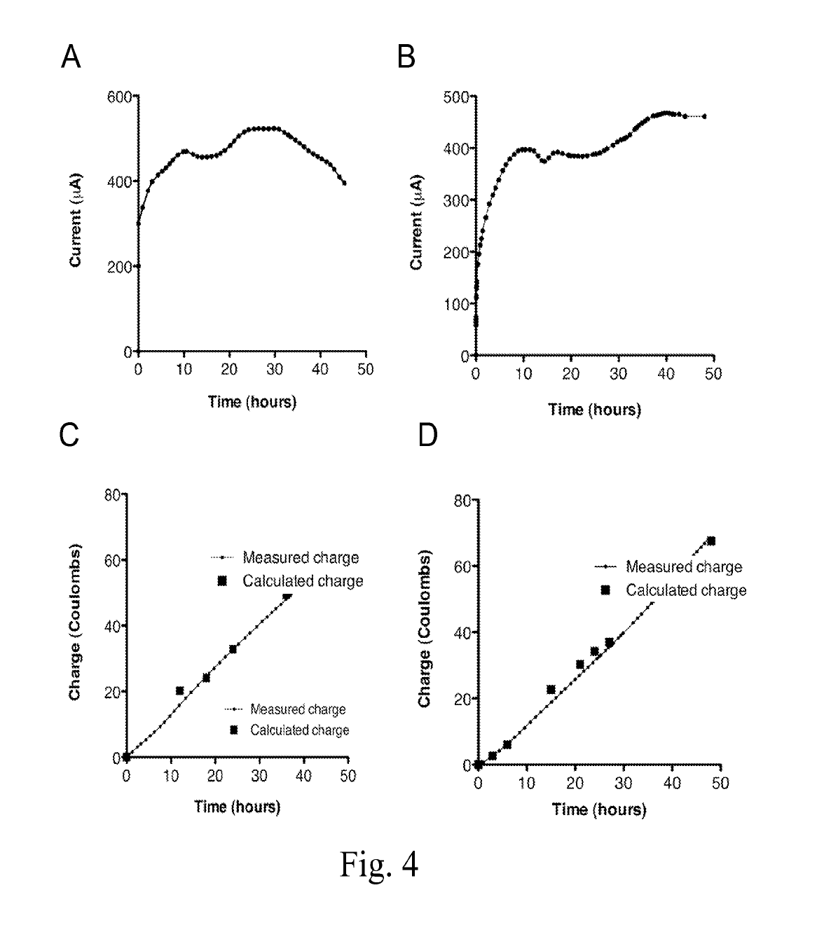

However, inoculation of S. oneidensis pGUT2PET (FIG. 4A) or Δpta pGUT2PET (FIG. 4B) into chambers containing electrodes poised at an oxidizing potential resulted in immediate anodic current flux, and consumption of glycerol (FIGS. 3B and 3C). Conversion of glycerol under these conditions was absolutely dependent upon the oxidizing electrode, demonstrating that this bioconversion was enabled by flux of electrons to the electrode. Moreover, when additional glycerol was added or when the medium was removed and replaced with fresh glycerol-containing medium, current production and glycerol consumption immediately resumed, showing that cells remained attached to the electrode and continuous operation was possible.

As observed in incubations with fumarate, only ethanol and a small amount of acetate accumulated during glycerol conversion; no succinate, malate, lactate, formate or other byproducts were detected for wild type (FIG. 3B) or Δpta (FIG. 3C) containing pGUT2PET. Mutants lacking pta exhibited a 46% decrease in acetate production compared to wild-type cultures when respiring to electrodes (FIG. 3C, Table 3). Continuous sparging with gas did not enhance conversion, nor was it essential for conversion of glycerol to ethanol in electrochemical reactors. To improve analytical measurements of coulombic yield by removing a potential electron donor (H₂) a low rate (1 ml/min) of gas sparging was employed in subsequent experiments. As this flushing also stripped some ethanol from the medium, volatile products were captured in a dry ice trap to confirm that ethanol was the only end product of glycerol bioconversion, as had been observed in our other experiments.

To further confirm that growth in electrochemical reactors led to stoichiometric glycerol to ethanol conversion, and eliminate the possibility that glycerol was being oxidized to CO₂ or other unknown compounds, a comparison between predicted and actual electron recovery of the engineered glycerol to ethanol pathway was performed. Electrochemical measurements record every electron equivalent excreted, allowing current output to be integrated over time (FIG. 4A and FIG. 4B), and compared with changes in glycerol and acetate levels over the same period. Electron amounts recorded agreed with the pathway in FIG. 1, where glycerol conversion to ethanol releases two electrons, and acetate production releases six electrons. Comparisons between predicted and measured charge transfer values in every experiment deviated no more than 10% (FIG. 4C), and the Δpta strain with pGUT2PET behaved similarly (FIG. 4D). As only endpoint measurements were possible via dry ice trapping of gas phase metabolites, ethanol production values at intermediate time points shown in FIGS. 3B and 3C are based on levels calculated from non-volatile metabolite measurements and columbic recovery. Taken together, the carbon and electron recovery data showed that the outcome of the engineered glycerol to ethanol pathway was identical when fumarate or an electrode was used as the electron acceptor.

The field of biocatilysis relies heavily on certain yeasts (Saccharomyces sp., Pichia sp., and Candida sp.) and bacteria (E. coli and Zymomonas sp.) to convert feedstocks into fuels (Dellomonaco et al., 2010 Microbial Cell Factories. 9:3). E. coli has been touted as an efficient and easily modifiable biocatalyst, with ethanol titers reaching 40 g/L (Yomano et al., 2008 Biotechnology Letters. 30(12):2097-2103) and volumetric productivities of strains engineered to ferment glycerol to ethanol with H₂ as a co-product as high as 4.7 mmoles/L/h (Yazdani and Gonzalez, 2008. Metabol. Eng. 10(6):340-351). With most microbe-electrode systems, the fact that the key reaction occurs at a surface makes electrode surface area a crucial factor. These experiments with engineered strains of Shewanella in reactors contained only 3 cm² electrode per ml, yet they already approached volumetric production rates of 1 mmol ethanol/L/h. High-surface area electrodes such as, for example, treated carbon brush electrodes, can easily achieve surface:volume ratios on the order of 30-70 cm²/ml (Watson and Logan, 2010 Biotechnol Bioeng. 105(3):489-498) and dramatically increase rates of current collection from Shewanella.

Electrode-linked microbial catalysis has many potential benefits: it can act as an electrochemical “lever” to drive an unfavorable reaction, allow generation of electricity via operation of a microbial fuel cell, or serve as reducing equivalents and/or oxidating equivalents for additional product synthesis. In the case of glycerol, the shift from aerobic oxidation (Trinh and Srienc, 2009 Appl. Environ. Microbiol. 75(20:6696-705) to anaerobic fermentation with formate or H₂ as an end product (Gonzalez et al., 2008 Metabol. Eng. 10(5):234-245; Dharmadi et al., 2006 Biotechnol. Bioeng. 94(5):821-829) represents a 3.5-fold decrease in the AG available to the cell. This low energy yield explains why anaerobic strategies are greatly enhanced by stripping of inhibitory byproducts such as H₂. In contrast, an oxidizing electrode can accelerate metabolism as it directly increases the thermodynamic driving force, and avoids oxidative losses (and costs) associated with oxygen (Trinh and Srienc, 2009 Appl. Environ. Microbiol. 75(21):6696-705). In a microbial fuel cell-like reactor, passing captured electrons to oxygen offers the possibility for −150 to −80 kJ/mol glycerol additional energy recovery, depending on the set potential of the anode. Alternatively, electrons can be boosted by a small potential (e.g., 0.25V to 0.5V) to power H₂- or CH₄-evolving microbial electrolysis chambers (Logan, 2009 Nat. Rev. Microbiol. 7(5):375-381; Liu et al., 2005 Environ. Sci. Technol. 39(11):4317-4320) at energetic yields superior to water-splitting reactors, as the bacteria supply the source electrons at a lower potential.

Though we have focused on a reductive electrobiocatalytic process, electrodes can also be used as electron donors for a variety of processes (Gregory et al., 2004 Environ Microbiol. 6(6):596-604; Gregory and Lovley, 2005 Environ Sci Technol. 39(22):8943-7; Strycharz et al., 2008 Appl Environ Microbiol. 74(19):5943-7; Wrighton et al., 2010. Isme J. 4(11):1443-1455) including a recent demonstration of acetogenesis by biofilms of Sporomusa ovata on graphite electrodes (Nevin et al., 2010 MBio. 1(2):e00103-10). Cathodic electrodes can drive biotransformation that require the input of electrons in electrode-interfaced biofilms of S. oneidensis.

To quantify inward electron flux, we have utilized electrode-attached S. oneidensis catalyzing the two-electron reduction of fumarate to succinate. S. oneidensis contains a soluble fumarate reductase localized to the periplasmic space, which is unique from the membrane-associated fumarate reductase in other bacteria (i.e., Escherichia coli) (Pealing et al., 1993 Biochemistry 32:3829-3829). Under anaerobic conditions, FccA is the sole fumarate reductase in Shewanella (Leys et al., 1999 Nat Struct Biol 6:1113-1117; Maier et al., 2003 J Basic Microbiol 43:312-327), and strongly favors the reductive reaction (Pealing et al., 1995 Biochem 34:6153-6158; Turner et al., 1999 Biochem 38:3302-3309). In the absence of soluble electron shuttles, contact between electron transfer proteins and the electrode surface is essential for electron transfer in S. oneidensis (Baron et al., 2009 J Biol Chem 284:28865-28873). This requirement, combined with sensitive electrochemical capabilities, was exploited to examine the hypothesis that the Mtr pathway could be reversed for cathodic electron uptake.

Using this electrochemical approach, combined with deletions of genes encoding cytochromes, structural proteins, and quinone biosynthesis, we determined that an intact OM protein complex (MtrCAB) can facilitate electrode-dependent fumarate reduction in S. oneidensis, and that the driving force required agrees with known potential-dependent responses of the fumarate reductase rather than the E^{ro} of the fumarate/succinate couple (+0.03 V). Furthermore, our data revealed a surprising requirement for CymA and the menaquinone pool for inward electron flux to periplasmic acceptors, and suggests a mechanism involving distinct CymA respiratory units for fumarate reduction (CymA:FccA) and metal reduction (CymA:MtrA). Taken together, our findings show a potential ability to drive pathways with electricity (electrosynthesis) in Shewanella using the Mtr respiratory pathway and provide mechanistic information about electron transfer into the cell.

Electrode-Dependent Fumarate Reduction by Thin Films of S. oneidensis Involves FccA.

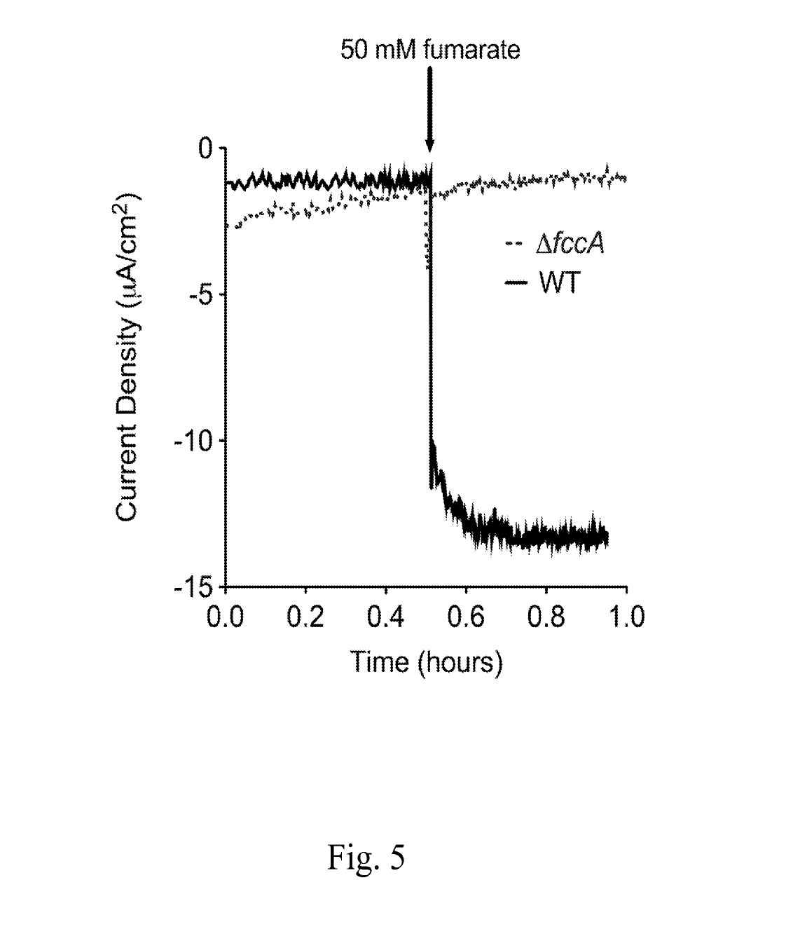

Our goal was to identify components necessary and sufficient for inward electron flux from an electrode surface into S. oneidensis. Using established electrochemical techniques [9B] thin films of S. oneidensis attached to a graphite electrode in the absence of added soluble shuttles were analyzed for their ability to catalyze the reduction of fumarate to succinate. When electrodes were poised at a reducing potential (−0.36 V versus SHE), continuous amperometry measurements showed a sudden onset of cathodic current upon addition of 50 mM fumarate to stirred anaerobic bioreactors, where wild type S. oneidensis reached an average net current density of −17.2±4.4 μA/cm² (n=6, FIG. 5). The observed negative current was indicative of electron flow from the electrode into attached cells. Furthermore, abiotic controls showed no electrochemical response upon fumarate addition. These experiments verified electron uptake capabilities of S. oneidensis attached to an electrode, and demonstrated the repeatability of the artificial biofilm technique.

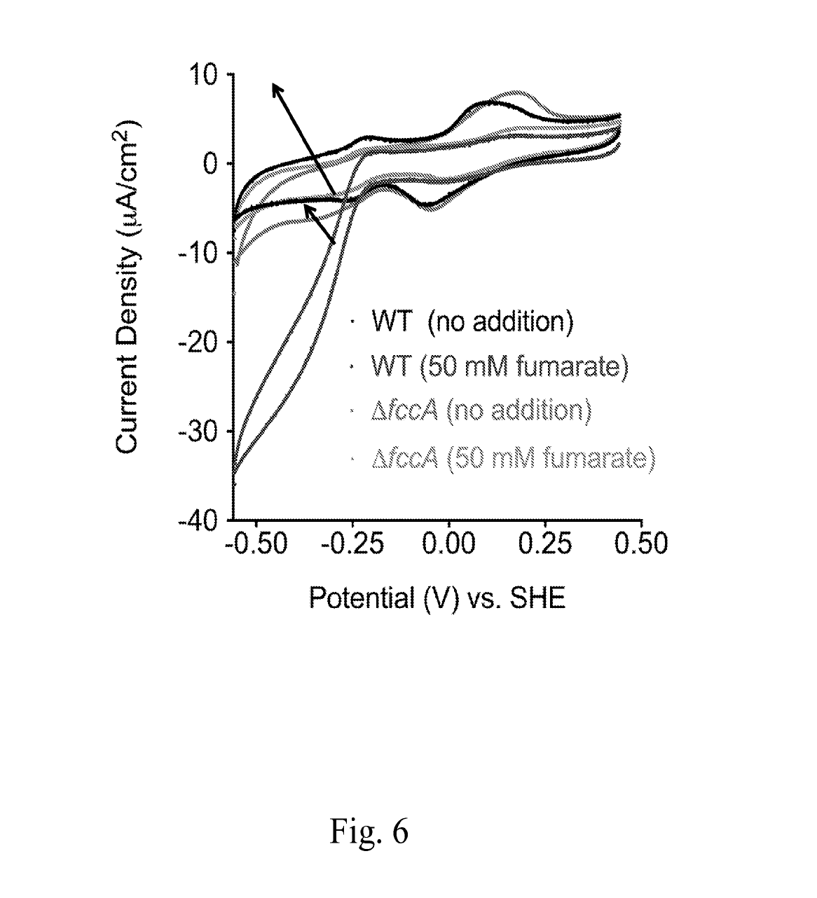

We next tested whether the observed electrochemical response was linked to the periplasmic fumarate reductase. In mutants lacking fccA, no sustained increase in current density was observed upon fumarate addition (FIG. 5), which further supported the conclusion that electrochemical responses were a direct measure of electron flow from the electrode into the periplasm for reduction of fumarate. To characterize electrode-linked fumarate reduction across a range of imposed potentials, slow scan rate cyclic voltammetry (CV) was performed in the presence and absence of fumarate (FIG. 6). Voltammograms of wild type or ΔfccA thin films in the absence of fumarate (single turnover) showed no significant differences, most notably in the potential range of outer membrane cytochromes (Baron et al., 2009 J Biol Chem 284:28865-28873; Firer-Sherwood et al., 2008 J Biol Inorg Chem 13:849-854). However, upon addition of fumarate a catalytic waveform with a midpoint potential centered at −0.26 V versus SHE, and a secondary boost at lower potentials (below −0.3 V) was observed in wild type cells. The fccA mutant showed no response, even at lower potentials (FIG. 6).

### Reversible Electron Transfer is Dependent Upon Outer Membrane and Periplasmic Cytochromes.

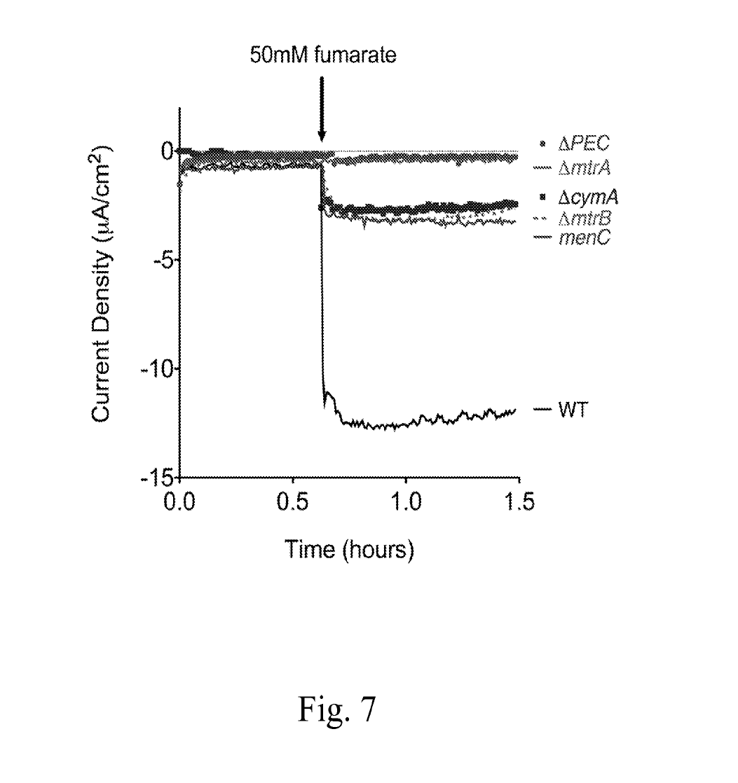

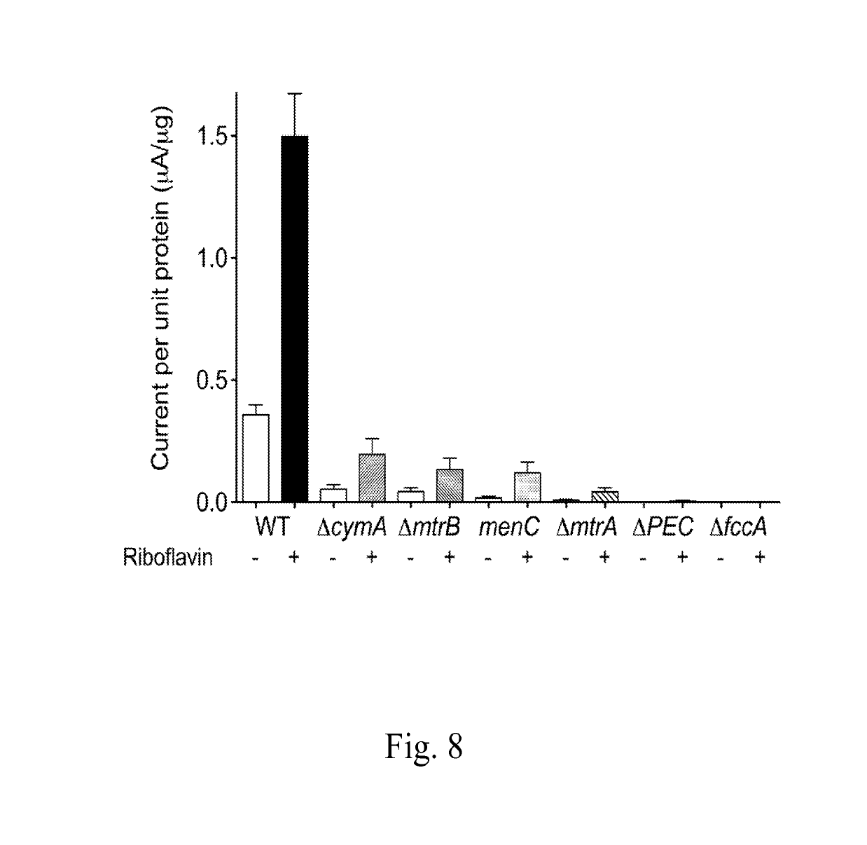

We next sought to investigate the role of the Mtr pathway in reversible electron transfer. Various mutants previously characterized for reduction of Fe(III) or soluble electron shuttles (Coursolle et al., 2010 J Bacteriol 192:467-474; Bretschger et al., 2007 Appl Environ Microbiol 73:7003-7012) were tested for their electrode-dependent fumarate reduction capabilities (FIG. 7). Since the Mtr pathway spans the cytoplasmic and outer membranes, we individually examined the importance of periplasmic and outer membrane components. Beginning with the outer surface, we exploited the fact that MtrB, a putative integral outer membrane β-barrel, is required for proper localization of MtrC and OmcA to the outer membrane (Myers and Myers, 2002 Appl Environ Microbiol 68:5585-5594). Deletion of the mtrB coding sequence eliminated most of the fumarate-dependent electron uptake current (FIG. 7). For a more quantitative assessment of electron transfer rates, all current values were normalized to total electrode-attached protein (μA/μg protein), to correct for differences in cell attachment between mutants. The mtrB mutant had a specific current that was 12% of the wild type rate (±1.7%, n=3, FIG. 8). These findings were consistent with previous studies showing MtrB is involved in outward electron transfer to electrodes (Coursolle et al., 2010 J Bacteriol 192:467-474; Bretschger et al., 2007 Appl Environ Microbiol 73:7003-7012).

To test the role of periplasmic components, soluble periplasmic cytochromes were deleted. In particular, two mutant strains were examined: ΔmtrA, and a mutant devoid of all known periplasmic electron carriers (mtrA, mtrD, cctA, dmsE, and SO4360) termed ΔPEC [32B]. Deletion of mtrA resulted in an almost complete loss of specific activity (3% of wild type, ±1%, n=3, FIG. 8). Likewise, ΔPEC showed even less electrodedependent fumarate reduction (0.4% of wild type, ±0.1%, n=3, FIG. 8). These mutants highlighted the need for a periplasmic electron carrier to complete the electrochemical circuit to FccA.

### The Majority of Electron Flux to FccA Proceeds Via CymA and the Menaquinone Pool.

If direct interaction between the periplasmic cytochrome MtrA and FccA is sufficient to direct electrons from the outer surface to FccA, cytoplasmic membrane-localized proteins would not be needed for inward electron flux. However, mutants lacking cymA were severely impaired in their ability to reduce fumarate. With a specific current of 15% of wild type (±3%, n=3), ΔcymA was not completely inactive, suggesting residual electron transfer between MtrA and FccA or MtrA and an unknown cytoplasmic membrane protein or periplasmic protein in vivo. Heme staining of whole cell extracts revealed no differences in cytochrome expression compared to wild type, confirming that the inability of a cymA mutant to reduce fumarate was due to the absence of CymA and not differential expression of the Mtr pathway. CymA has only been shown to be required for electron flow from dehydrogenases in the cytoplasmic membrane to periplasmic enzymes (e.g., FccA) (Myers and Myers, 1997 J Bacteriol 179:1143-1152; Myers and Myers, 2000 J Bacteriol 182:67-75; Schwalb et al., 2002 Biochem Soc Trans 30:658-662), yet our data show that under these conditions, the route of electron flow from the outer surface to periplasmic fumarate reductase still involved cytoplasmic membrane components.

In S. oneidensis, the menaquinone pool links primary dehydrogenases (i.e., formate or NADH dehydrogenase) to CymA (Myers and Myers, 1997 J Bacteriol 179:1143-1152; Myers and Myers, 2000 J Bacteriol 182:67-75). The fact that CymA was responsible for 85% of the total current flowing from electrodes into the cell for fumarate reduction, combined with its known interaction with the menaquinone pool, suggests a possible role for the menaquinone pool in linking electron flux back out to another CymA protein, and finally to FccA (FIG. 8). A previously characterized mini Tn-10 insertion in the menC coding sequence (o-succinylbenzoate synthase (Newman and Kolter, 2000 Nature 405:94-97)), which is required for menaquinone biosynthesis (Guest, 1977 J Bacteriol 130:1038-1046; Sharma et al., 1993 J Bacteriol 175:4917-4921) was used in these studies. The specific current of menC mutant cells was only 5% (±2%, n=3) compared to wild type (FIG. 8). Heme stain profiles of whole cell extracts separated by SDS-PAGE showed that expression of CymA and other Mtr proteins was not affected by the menC insertion. Taken together, these data show that the majority of inward electron flux from the electrode to fumarate reductase involves both outer membrane and periplasmic components of the Mtr respiratory pathway, and passed into the menaquinone pool before re-entering the periplasm.

### Riboflavin Enhances Electrode-Dependent Inward Electron Flux to FccA but Cannot Replace Lost Periplasmic or Cytoplasmic Components.

Previous experiments have shown that soluble redox shuttles (e.g., riboflavin, FAD, and FMN) enhance turnover rates of cytochromes reducing insoluble iron oxides, even at the 0.5-1 μM levels that typically accumulate in Shewanella planktonic cultures (Ross et al., 2009 Appl Environ Microbiol 75:5218-5226; von Canstein et al., 2008 Appl Environ Microbiol 74:615-623) and electrode-attached biofilms (Coursolle et al., 2010 J Bacteriol 192:467-474; Baron et al., 2009 J Biol Chem 284:28865-28873; Marsili et al., 2008 Proc Natl Acad Sci USA 105:3968-3973). Similar to what has been observed with pure cytochromes (Ross et al., 2009 Appl Environ Microbiol 75:5218-5226) and whole cells (Baron et al., 2009 J Biol Chem 284:28865-28873), addition of riboflavin (1 mM) to wild type cells stimulated electron flux over 4-fold, with an average current density of 1.5±0.3 μA/μg (FIG. 4). However, addition of 1 μM riboflavin to ΔmtrB only raised the rate of fumarate-specific electron uptake to 0.13±0.05 μA/μg (FIG. 8). The inability of riboflavin to restore ΔmtrB underscores the involvement of the outer membrane conduit in delivering electrons to the periplasm, and showed that soluble shuttling from the electrode into the periplasm could not replace this direct pathway, even at concentrations of 1 μM.

Another interaction that could have been enhanced by flavin shuttling was the slow rate of electron transfer attributed to MtrA-FccA remaining in ΔcymA and the menC mutant. Addition of 1 μM riboflavin to ΔcymA and the menC mutant had a larger stimulatory effect on the rate of electron uptake compared to ΔmtrB, but remained well below the current density observed in wild type cells with riboflavin (FIG. 8). Thus the electron uptake rate for ΔcymA and the menC mutant (approximately 20% of wild type) could represent an upper boundary for electron transfer between MtrA and FccA in the presence of physiological flavin levels. Adding riboflavin to periplasmic cytochrome mutants (ΔmtrA, ΔPEC) had no stimulatory effect; further supporting the model that riboflavin shuttling from electrodes into the periplasm could not significantly contribute to the overall reaction.

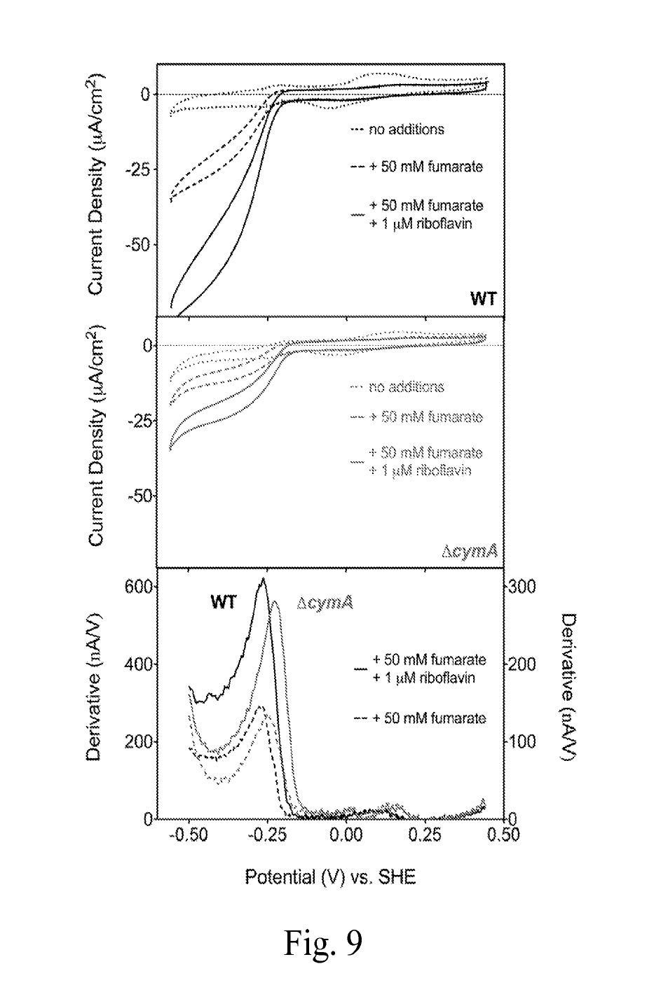

A final analysis aimed at elucidating the role of flavins in electron uptake was cyclic voltammetry. Adding 1 μM riboflavin to wild type cells reducing fumarate increased the limiting current, but did not alter the onset potential or midpoint potential of the catalytic wave (FIG. 9). Thus, electrochemical responses were consistent with an increased turnover of the overall pathway (e.g., electrode-to-outer membrane conduit), and the shape of the wave reflected no significant change in the driving force required to reduce FccA. Voltammetry of ΔcymA in the presence of flavins confirmed the weak stimulation observed in poised-potential experiments (FIG. 9), but also revealed a shift in the potential dependence of the catalytic reaction. The midpoint potential of the catalytic wave increased by +50 mV when 1 μM riboflavin was added to biofilms actively reducing fumarate; the menC mutant exhibited a similar shift. The positive shift in potential further suggested that flavins enabled electron transfer to FccA, in the absence of CymA, via a pathway unique from what was active in wild type cells.

In this work, voltammetric techniques typically used to examine electron transfer from bacteria to surfaces (Marsili et al., 2008 Appl Environ Microbiol 74:7329-7337; Marsili et al., 2008 Proc Natl Acad Sci USA 105:3968-3973; Richter et al., 2009 Energy Env Sci 2:506-516; Srikanth et al., 2008 Biotechnol Bioeng 99:1065-1073) were used to demonstrate the ability of electrode surfaces to drive reductive reactions in S. oneidensis. Comparison of multiple mutants provided evidence for the role of specific proteins from the Mtr respiratory pathway in this process. The main pathway for outward electron flux to insoluble metals, electron shuttles, and electrodes involves the MtrCAB protein complex, which includes MtrC and MtrA interacting through MtrB (Hartshorne et al., 2009 Proc Natl Acad Sci USA 106: 22169-22174). Interaction between MtrC and MtrA, and consequently facile electron transfer across the outer membrane, has never been shown in the absence of MtrB, since MtrB is required for the proper localization of MtrA (Hartshorne et al., 2009 Proc Natl Acad Sci USA 106: 22169-22174), MtrC and OmcA (Myers and Myers, 2002 Appl Environ Microbiol 68:5585-5594). In our electron uptake assay ΔmtrB exhibited approximately 12% of the activity of wild type, illustrating the primary role of this conduit.

In addition to MtrCAB, the S. oneidensis genome encodes modular paralogs (i.e., MtrDEF) of the Mtr respiratory pathway (Coursolle and Gralnick, 2010 Mol Microbiol 77:995-1008). As such, residual activity in ΔmtrB may be due to either individual Mtr pathway paralogs, or to complete alternative complexes in the membrane. For example, MtrB paralogs such as MtrE can partially rescue ΔmtrB. An alternative hypothesis is that a complete conduit comprised of MtrDEF could be responsible for the remaining activity in ΔmtrB.

Deleting periplasmic components provided evidence against the complete MtrDEF conduit hypothesis, as mutants lacking mtrA were capable of only about 3% of wild type rates. This demonstrated that only 3% of electron uptake could be attributed to alternative complexes such as, for example, MtrDEF. The low residual effect of other periplasmic cytochromes mtrD, cctA, dmsE, and SO4360 was also consistent with recent findings (Coursolle and Gralnick, 2010 Mol Microbiol 77:995-1008) where MtrD, CctA and DmsE were found to play a minor role in insoluble iron reduction in the absence of primary periplasmic electron carriers (i.e., MtrA). Furthermore, while a stable MtrAB subcomplex can form in the absence of MtrC, MtrB does not form a complex with MtrC in the absence of MtrA (Hartshorne et al., 2009 Proc Natl Acad Sci USA 106: 22169-22174). Thus, MtrA is involved in rapid electron transfer into the periplasm, primarily as part of the MtrCAB complex, and other outer membrane-spanning complexes do not play a major role under these conditions.

The involvement of CymA and the menaquinone pool for electrode-dependent fumarate reduction was determined through mutant analysis and was corroborated by thermodynamic evidence. In slow scan rate CV analysis, the catalytic waveshape from wild type films (FIG. 6) was similar to purified protein films of FccA (Butt et al., 2000 Biophysical Journal 78:1001-1009; Morris et al., 1994 Biochem J 302:587-593) with an onset potential and secondary boost well below the fumarate/succinate redox couple. In wild type films, however, the midpoint potential was centered at −270 mV versus SHE (FIG. 9), nearly 100 mV more negative than what is required to drive reduction by purified FccA. The fact that disruption of CymA and menaquinone biosynthesis shifted the onset and midpoint potential of fumarate reduction more positive (FIG. 9) indicated that electron flow through CymA and the menaquinone pool was partially responsible for this stronger driving force requirement.

In vitro kinetic assays have demonstrated that direct and reversible electron transfer can occur between purified MtrA and FccA (Schuetz et al., 2009 Appl Environ Microbiol 75:7789-7796). However, rapid electron transfer between CymA and MtrA was also observed, and was determined to be 1.4-fold faster than the MtrA:FccA couple. Our study revealed a similar bias favoring the CymA:MtrA pathway and supports a model where the main conduit into the cell prefers MtrA reducing CymA, and that in vivo MtrA reduces FccA as a secondary reaction (<15% of activity), either by direct transfer or via periplasmic intermediates. For example, riboflavin was able to accelerate electron transfer in a ΔcymA strain (FIG. 8). Further evidence that flavins were altering the overall pathway was found in the shift in driving force needed to reduce fumarate when riboflavin was provided to the cymA mutant. This involvement of MtrA also confirmed that FccA was unable to be reduced by riboflavin shuttling from the electrode and across outer membrane at appreciable rates.

While the Mtr pathway in S. oneidensis is involved in electron transfer both into and out of the cell, this contrasts with recent data suggesting Geobacter sulfurreducens exploits two separate pathways (Strycharz et al., 2011 Bioelectrochemistry 80:142-150). Gene deletions and gene expression studies suggest that electron transfer into Geobacter biofilms is independent of major outer membrane cytochromes (OmcZ, OmcB, OmcST, and OmcE) required for electron transfer out of the cell, and was instead dependent upon a putative monoheme c-type cytochrome (Strycharz et al., 2011 Bioelectrochemistry 80:142-150).

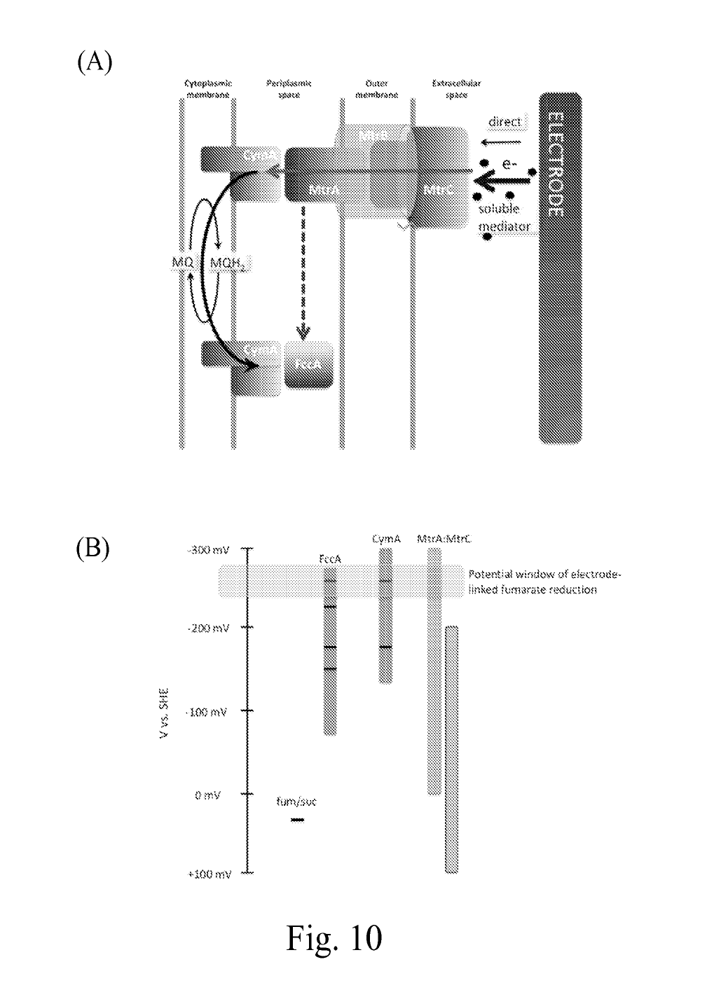

Here we report genetic, electrochemical, and thermodynamic evidence for the reversibility of the Mtr electron transfer pathway in S. oneidensis with the involvement of CymA and the menaquinone pool in electrode-dependent reduction of fumarate by whole cells. Examination of reverse electron flow driven by an electrode also afforded mechanistic insights to periplasmic electron transfer in S. oneidensis, supporting distinct respiratory units comprised of CymA:MtrA and CymA:FccA in vivo, illustrated in FIG. 10. The ability to use electrodes for extracellular reduction of the quinone pool suggests the potential to catalyze net reductions within the cell, which would also consume intracellular protons, and generate a proton motive force. The fact that the Mtr conduit alone can create this linkage, combined with recent functional expression of MtrCAB in E. coli (Jensen et al., 2010 Proc Natl Acad Sci USA 107:19213-19218) suggests a role for this pathway in engineered microbial electrosynthesis (Rabaey and Rozendal, 2010 Nature Reviews Microbiology 8:706-716).

It has been previously proposed that electrodes could alter fermentative pathways, but these strategies required high concentrations of toxic redox mediators to extract electrons from fermentative cells unable to transfer electrons to their outer surface for electrode respiration (Emde et al., 1989 Appl. Microbiol. Biotechnol. 32(2):170-175; Park and Zeikus, 1999 J. Bacteriol. 181(8):2403-10; Zhang et al., 2010 Appl. Environ. Microbiol. 76(8):2397-2401). S. oneidensis proved a tractable organism to link native electron transfer ability to synthetic biology. Our work here shows that the landscape of metabolic engineering and synthetic biology strategies for biofuel and bioproduct synthesis (Clomburg and Gonzalez, 2010. Appl. Microbiol. Biotechnol. 86(2):419-434) can be expanded through the use of engineered electrode-interfaced bacteria.

While we have demonstrated the feasibility of electrode-dependent metabolism using S. oneidensis, electrode-dependent metabolism may be feasible using any microbe capable of exchanging electrons with an electrode. The ability of a microbe to do so is typically linked to metal reduction and oxidation. Thus, electrode-dependent metabolism may be performed using microbes linked to metal reduction and oxidation, referred to herein generically as “electron flux microbes.” Such microbes include, for example, members of the genera Geobacter, Pelobacter, Desulfuromonas, Desulfuromusa, Geothermobacter, Geopsycrobacter, Anaeromyxobacter, Desulfovibrio, Desulfobulbus, Geothrix, Clostridium, Deferribacter, Acidomicrobium, Acidithiobacillus, Aeromonas, Bacillus, Desulfitobacterium, Desulfosporosinus, Sporomusa, Rhodoferax, Rhodopseudomonas, Ferrimonas, Ferriglobus, Geoglobus, Gallionella Geothermobacter, Geothermomicrobium, Geovibrio, Pantaea, Pyrobaculum, Thermotoga, Pyrodictium, Sulfobacillus, Sulfospirillum, Shewanella, Sideroxidans, Thermoanaerobacter, Thermococcus, Thermus, Trichlorobacter, Dechloromonas, Azospira, Pseudomonas, Ochrobacterium, Acidiphilum, Therminocola, Vibrio, Marinobacter, Leptothrix, Rhodobacter, Rhodovulum, Chlorobium, Thiodictyon, Mariprofundus, and combinations of such microbes. In some embodiments, electrode-dependent metabolism may be performed using a member of the genus Shewanella.

While the description that follows is presented in the context of using Shewanella oneidensis as an electron flux microbe, other embodiments of the devices, methods, or microbes described herein can exploit other electron flux microbes. As various members of the Geobacteraceae family have been shown to transfer electrons from wide range of compounds (including glycerol, benzoate, phenol, ethanol, lactate, acetate, malate, amino acids, palmitate, citrate, and methanol) to extracellular acceptors, similar redox balancing approaches can be conducted with representatives of this group. Oxidation of these carbon and electron sources produce a variety of reducing equivalents including NADH, NADPH, menaquinones and ferredoxin, showing that metabolic pathways utilizing these compounds can be interfaced with electrodes (via extracellular electron transfer) using the native electron transfer pathways. For effective production of value added end compounds, the approach would only require elimination of complete citric acid cycle function using conventional methods such as, for example, deletion of gltA (citrate synthase), por (pyruvate ferredoxin oxioreductase), oor (2-oxoglutarate ferredoxin oxioreductase), or sfrAB (putative NADPH oxioreductase) combined with expression of synthesis pathways (such as the pdc/adh module used in Shewanella). Multiple studies have demonstrated transfer and replication of the pBBR1MCS-based plasmids used in Shewanella, showing its utility in development of such pathways.

Mutants defective in the TCA cycle can be constructed and grown using conventional methodology. Simple insertional deletions are achieved in Geobacter by amplifying ˜500 bp regions up- and downstream of the gene to be deleted, and fused to antibiotic resistance cassettes using complementary primer overhangs. After electroporation of linear fragments into competent cells, selection with the appropriate antibiotic under anaerobic conditions selects clones in which double recombination events have replaced the gene of interest with the antibiotic cassette. For subsequent removal of the gene encoding antibiotic resistance, two protocols are used. Either the linear fragment is constructed with FRT sequences recognized by flp recombinase, and the strain is later transformed with a counterselectable plasmid expressing the recombinase (such as pFLP2), or the original gene replacement is conducted using a fragment of up- and downstream DNA, cloned into a counterselectable plasmid (such as pSMV3) that is inserted into the genome via a single recombination event. These genetic backgrounds can be verified to be defective in TCA cycle function by demonstrating a lack of growth with acetate as the sole carbon an energy source, and growth with hydrogen as an energy source, and supplemental acetate as a carbon source. These mutants can then serve as hosts for recombinant metabolic pathways using either genomic insertions, or transformation with commonly utilized plasmid backbones such as pBBRMCS-1 or pRG5.

In other embodiments, electrode-dependent metabolism may be performed using microbes that contain one or more heterologous nucleic acid molecules that includes a coding sequence from one or more electron flux microbes that encodes a protein associated with metal reduction and/or oxidation. Thus, electrode-dependent metabolism may be performed using, for example, E. coli or S. cerevisiae modified to include one or more coding regions of, for example, the Shewanella Mtr pathway.

The microbes used to perform electrode-dependent metabolism can be genetically modified to include one or more coding sequences derived from a heterologous organism. FIG. 1 illustrates one exemplary embodiment in which S. oneidensis is modified to include coding sequences derived from E. coli and Zymomonas mobilis. In the embodiment illustrated in FIG. 1 as well as other embodiments, heterologous coding sequences can be provided in a module, a collection of two or more coding regions so that the module provides coding sequences that encode enzymes that catalyze one or more metabolic steps. In the embodiment illustrated in FIG. 1, a glycerol module includes four coding sequences derived from E. coli that encode E. coli enzymes involved ion glycerol metabolism. The embodiment illustrated in FIG. 1 also includes an ethanol module that includes two coding sequences derived from Z. mobilis that encode Z. mobilis enzymes involved in ethanol synthesis. In an alternative embodiment, an alternative glycerol module can include glpK and gldA coding regions from E. coli. The enzymes encoded by these coding regions are sufficient, when introduced into S. oneidensis, to convert glycerol to dihydroxy acetone, which may then feed into native S. oneidensis metabolism (see FIG. 1).

In some embodiments, the particular heterologous coding sequences may be selected based on the substrate to be provided to the microbe, the desired end product, and the native biochemistry of the microbe. In the embodiment illustrated in FIG. 1, S. oneidensis is unable to natively utilize glycerol or to natively produce ethanol. Thus, the S. oneidensis was modified to include E. coli-derived coding sequences that encode E. coli enzymes involved in glycerol metabolism and Z. mobilis-derived coding sequences that encode Z. mobilis enzymes involved in ethanol synthesis. The embodiment illustrated in FIG. 1 also includes heterologous coding sequences such as, for example, tpiA, which creates a metabolic bridge between the glycerol module and the native S. oneidensis Mtr pathway.

Heterologous coding sequences may be provided on one or more plasmids that may be introduced into the host microbe using methods routine in the field of molecular biology. When the heterologous coding sequences are organized into modules, one or more modules may be provided on a single plasmid. When appropriate, a module may be provided on one or more than one plasmid.

Electrode-dependent metabolism has broad applicability. Consequently, it is impractical to expressly describe each and every combination of redox-imbalanced substrate and product. However, the full scope of the applicability of electrode-dependent metabolism may be illustrated through the embodiment illustrated in FIG. 1 and description of alternative embodiments herein.

A non-exhaustive list of exemplary substrates includes, for example, hexoses (e.g., glucose, mannose, galactose, fructose, etc.), pentoses (e.g., arabinose, xylose, etc.), glycerol, fatty acids, lactate, mixed hydrocarbons (e.g., decane, undecane, dodecane, tridecane, tetradecane, pentadecane, hexadecane, heptadecane, octadecane, nonadecane, eicosane, pristane, cyclohexane, naphthalene, cumene, etc.), and organic acids (e.g., lactate, acetate, citrate, etc.). When the end product is more oxidized than the substrate, the electrode can serve as an electron sink. When the product is more reduced than the substrate, the electrode can serve as an electron source.

The use of electrodes to balance a metabolic pathway utilizing a particular substrate depends upon the relative redox states of the substrate and product. Typically, fermentations have been limited to either perfect matches between substrate and product (these are rare, one example is glucose to ethanol), cases where making more than one product is tolerable (glycerol to ethanol and formate), or oxygen is added to balance an oxidation (glycerol to ethanol plus some CO₂). Electrode-dependent metabolism allows the conversion of a substrate to a broader spectrum of products, independent of the relative redox states of the substrate and product.

Because our system is reversible, the spectrum of potential products includes those in which the product is in a reduced redox state compared to the substrate. Conventionally, sources of electrons include fermentation feedstocks and/or the splitting of water in, for example, photosynthesis. Using feedstock as a source of electrons in conventional reductive fermentations can reduce yield. In the case of photosynthesis, the reactions require light, which can be difficult to deliver efficiently. Our system allows one to directly add electrons to the reaction, which can provide more efficient use of the carbon feedstock (e.g., substrate) and circumvent the requirement for light for certain reductive reactions using, for example, CO₂ as a carbon substrate.

A non-exhaustive list of exemplary products that may be either more oxidized or more reduced than the substrate includes, for example, alcohols (e.g., ethanol), lactate, acetate, succinate, malate, citrate, 1,3-propanediol, ascorbic acid, shikimic acid, 3-hydroxypropanoic acid, and dihydroxyacetone. Biopolymers such as polyhydroxyalkanoate, polyhydroxybutyrate, and polyhydroxyvalerate, can also be produced, primarily via reductive reactions. Fuel-like products that are typically at a higher degree of reduction than sugars and glycerol include, for example, isopropanol, 1-butanol, butanol, 2-methyl-1 butanol, isopentanol, fatty alcohols, and olefins.

The products may be commercial-ready (e.g., certain fuel-like products) or may be a component of, an ingredient in the production of, or chemical intermediate in the production of, another product. For example, dihydroxyacetone (DHA) is used in sunless tanning products. 1,3-propanediol is a chemical base for wood paint and polyesters. Succinate can be used as a feedstock for production of many bulk chemicals such as, for example, 1,4-butanediol, tetrahydrofuran, succinate salts (for deicers), and adipic acid (for nylon). n-butanol is used industrially for pharmaceutical manufacturing, pyroxylin plastics, and polymers. 3-hyroxybutyrate can be utilized to make biodegradable plastics.

Other exemplary products include, for example, organic acids such as, for example, malonic acid, glucaric acid, itaconic, acid, and hydroxyl-citric acid.

Table 1 provides exemplary redox-imbalanced pathways, the balanced redox reaction for each pathway, exemplary coding sequences that may useful for constructing a modified host microbe, and exemplary source organisms for the exemplary coding sequences.

TABLE 1

| | | | |
|---|---|---|---|
| | | Coding | |
| Pathway | e− Balanced Reaction | sequences | Source |

OXIDATIONS

| | | | |
|---|---|---|---|
| Glycerol → | C₃H₅(OH)₃ → C₃H₆O₃ + 2e⁻ | glpF, gldA | Escherichia coli |
| dihydroxyacetone | | | |
| Glycerol → | C₃H₅(OH)₃ → C₃H₆O₃ + 2e⁻ | glpF, sldAB | Escherichia coli, |
| dihydroxyacetone | | | Gluconobacter oxydans |
| Glucose → malonate | C₆H₁₂O₆ → 2 CH₂(COOH)₂ + 8e− | accABCD, tesA | Escherichia coli |
| | | (targeted to | |
| | | cytoplasm) | |
| Glucose → glucaric | C₆H₁₂O₆ + 2e− → C₆H₁₀O₈ | Inol, MIOX, | Saccharomyces |
| acid (saccharic acid) | | Udh | cerevisiae, mouse, |
| | | | Pseudomonas syringae |

REDUCTIONS

| | | | |
|---|---|---|---|
| Glycerol → 1,3- | C₃H₅(OH)₃ + 2e⁻ → C₃H₈O₂ | dhaB1, dhaB2, | Clostridium butyricum |
| propanediol | | dhaT | |
| Glucose → | C₆H₁₂O₆ + 4e⁻ → 2 (C₄H₆O₄) | pepC, mqo, | Anaerobiospirillum |
| 2 succinate | | fumAB, frdABC | succiniciproducen, |
| | | | Escherichia coli |
| 2 Acetate → 3- | 2 C₂H₄O₂ + 2e⁻ → C₄H₈O₃ | phbC, phbA, | Alcaligenes eutophus |
| hydroxybutyrate | | phbB | |
| Glycerol → n-butanol | C₃H₅(OH)₃ + CO₂ + 10e⁻ → C₄H₁₀O | alsS, ilvC, ihD, | Lactococcus lactis, |
| | | kivd, adh | Escherichia coli |

In some embodiments, the heterologous coding sequences may be selected based on the native ability of the microbe to perform electrode-dependent metabolism. For example, E. coli is unable to naturally transfer electrons to extracellular minerals (e.g., an electrode) or soluble redox shuttles. Coding sequences that express one or more proteins involved in extracellular transfer of electrons may be introduced into, for example, E. coli so that the resultant modified organism possesses at least some of the native metabolic capabilities of E. coli, but is further capable of transferring intracellular electrons to an extracellular substrate.

The plasmid constructs, modified microbes, and methods described herein are not dependent upon any particular set of coding regions or the identity of any particular source organism—i.e., genus, species, or strain. Thus, particular coding sequences described herein are merely exemplary. Those of ordinary skill in the art are fully capable of identifying coding sequences that are variants of those described herein but would be expected to provide similar function. Modern genetic databases, bioinformatics, and other techniques can allow one to identify suitable analogs and or variants in other strains of a particular species or in entirely different species that can be introduced into a heterologous cell, be expressed, and provide the desired biological function. For example, many alcohol dehydrogenases have been identified, characterized, cloned, and sequenced. Thus, while the exemplary embodiment shown in FIG. 1 includes an ethanol module that includes an E. coli adhB coding sequences, it is expressly contemplated that alternative embodiments can include any suitable alcohol dehydrogenase from any source.

We have determined the necessary and sufficient components of the S. oneidensis Mtr respiratory pathway required for linking intracellular metabolism to reduction of extracellular substrates. We have systematically deleted all proteins of the Mtr respiratory pathway from S. oneidensis MR-1 and functionally expressed CymA, MtrA, MtrB, and MtrC in the mutant background. Moreover, we have taken the mtrCABcymA construct and engineered E. coli into an electrode-respiring organism. Here, we present a minimal set of genes from S. oneidensis capable of electron transfer to extracellular iron and carbon electrodes with implications for bolstering new metabolic products with minimal system engineering.

The MtrCAB/CymA electron transfer module can restore Fe(III) reduction in a Shewanella strain lacking all periplasmic and outer membrane cytochromes.

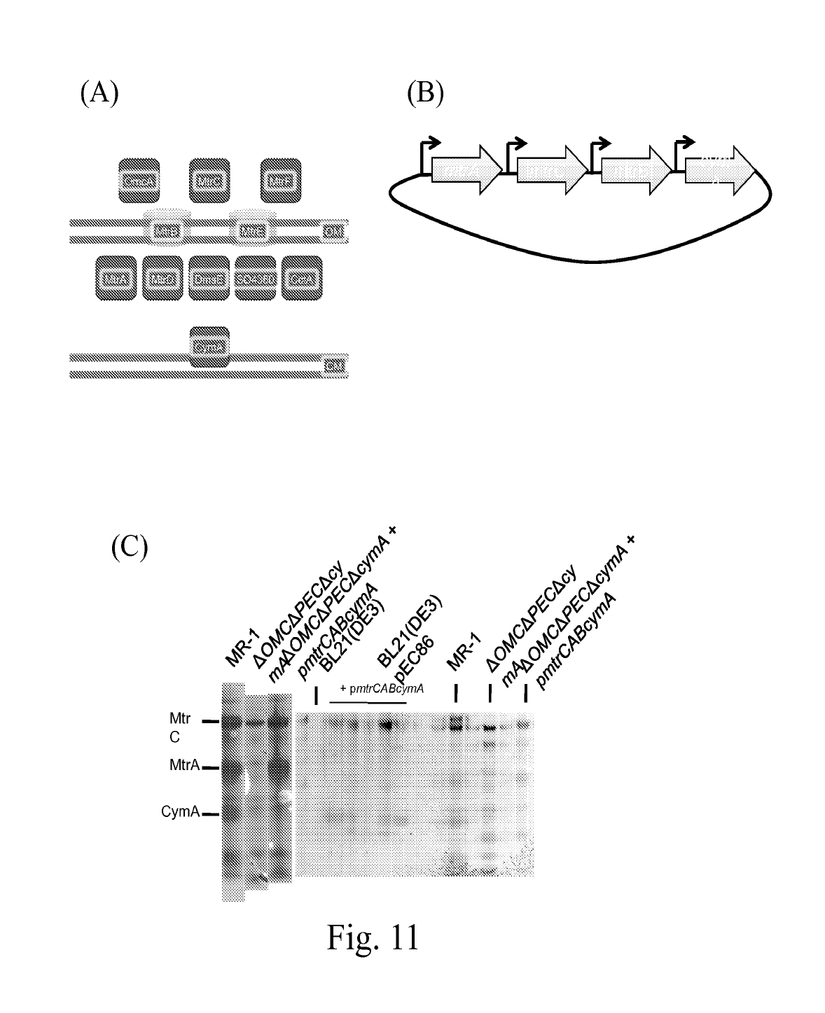

In S. oneidensis MR-1, deletion mutants have shown that reduction of Fe(III) is most severely affected by the absence of CymA, which transfers electrons from the quinone pool to the periplasm, and MtrCAB, which form a conduit allowing electrons to travel across the outer membrane (Coursolle and Gralnick, 2010 Mol Microbiol 77:995-1008; Ross et al., 2009 Appl Environ Microbiol 75:5218-5226; Hartshorne et al., 2009 Proc Natl Acad Sci USA 106: 22169-22174; Baron et al., 2009 J Biol Chem 284:28865-28873). However, as the genome of S. oneidensis also encodes multiple paralogues of mtrC, mtrA, and mtrB, such as the cluster containing mtrF, mtrD, and mtrE, residual Fe(III) reduction capacity in MtrCAB mutants can be detected (Coursolle and Gralnick, 2010 Mol Microbiol 77:995-1008; Coursolle and Gralnick, “Reconstruction of extracellular respiratory pathways for iron(III) reduction in Shewanella oneidensis strain MR-1,” 2011, Submitted). To study the minimal set of coding sequences sufficient for extracellular electron transfer in the absence of these effects, we engineered a new strain of Shewanella containing 11 markerless deletions, and was devoid of all major outer membrane c-type cytochromes (OmcA, MtrC, MtrD), Mtr-like outer membrane β-barrel proteins (MtrB, MtrE, DmsE), all periplasmic c-type cytochromes (MtrA, MtrF, SO4360, CctA), and the link to the quinone pool (CymA) (FIG. 11A). This strain was denoted ΔOMC/ΔPEC/ΔcymA.

A single plasmid containing the outer membrane electron transfer conduit (mtrC, mtrA, and mtrB) along with the coding sequence encoding the inner membrane tetraheme cytochrome CymA was then constructed. Each coding sequence was placed under control of its own lac promoter, to create pmtrCABcymA (FIG. 11B). To verify expression levels of each protein, the plasmid was transferred into the mutant strain lacking all inner and outer membrane cytochyromes (ΔOMC/ΔPEC/ΔcymA), and cell extracts were analyzed for the presence of MtrC, MtrA, and CymA using heme staining (Thomas et al., 1976 Anal. Biochem. 75:168-176), while MtrB was detected using immunoblot analysis (FIG. 11C). These experiments showed similar levels of cytochromes were present in the complemented strain when compared to the wild type.

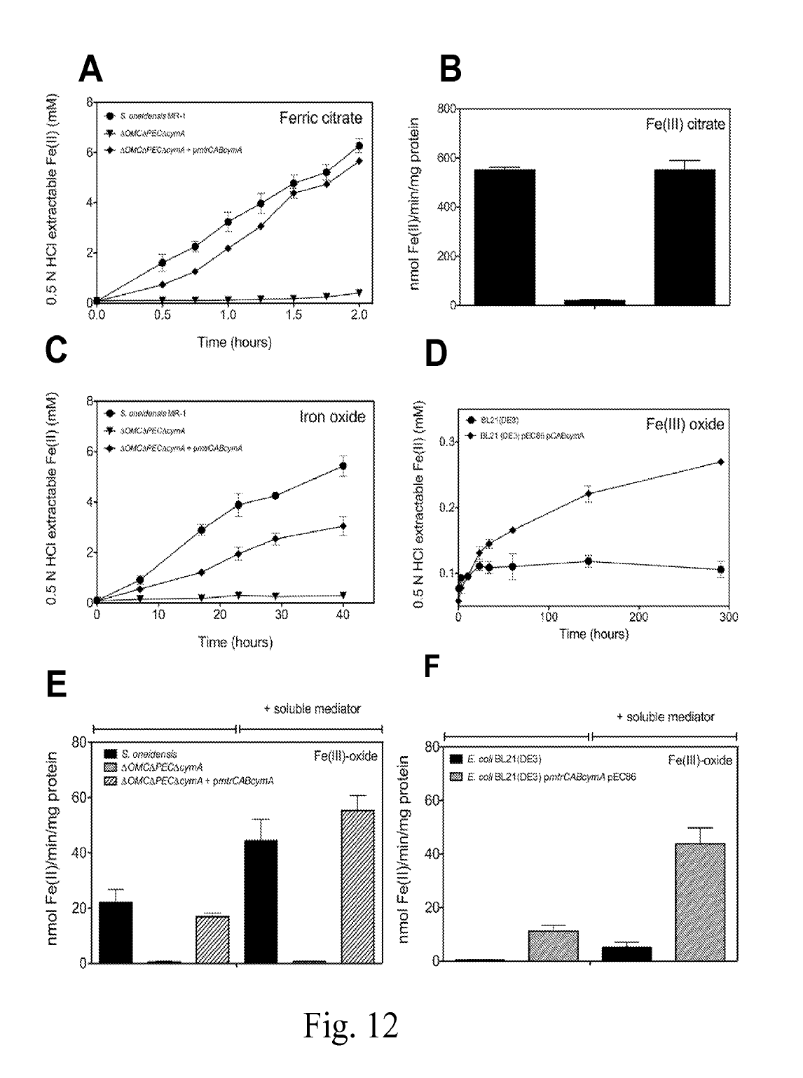

Next, the ability of the pmtrCABcymA module to restore Fe(III)-reduction ability to S. oneidensis MR-1 was tested. Washed S. oneidensis MR-1, S. oneidensis ΔOMC/ΔPEC/ΔcymA, or S. oneidensis ΔOMC/ΔPEC/ΔcymA+pmtrCABcymA cells (all at OD₆₀₀ 0.13) were incubated with Fe(III) citrate under anaerobic conditions. While S. oneidensis reduced Fe(III) citrate at a rate of 575 nmol Fe(II) mg protein⁻¹ min⁻¹, the 11-coding sequence deletion strain reduced soluble Fe(III) at less than 1% of this rate, confirming the complete removal of all electron transfer components. When the four-coding sequence construct was introduced into this same strain, Fe(III)-citrate reduction returned to wild-type rates (FIG. 12A,C). These results were consistent with conclusions from mutant and biochemical studies (Myers and Myers, 1997 J Bacteriol 179:1143-1152; Beliaev et al., 2001 Mol Microbiol 39: 722-730; Büucking et al., 2010 FEMS Microbiol. Lett. 306:144-151; Coursolle and Gralnick, 2010 Mol Microbiol 77:995-1008).

Similar experiments were conducted to determine if pmtrCABcymA was sufficient to restore electron transfer to an insoluble electron acceptor, Fe(III) oxide. Again, the 11-coding sequence deletion completely eliminated electron transfer to Fe(III), while complementation with the pmtrCABcymA significantly restored activity to within 80% of wild-type rates. (FIG. 12C, Table 6). As deletion of the outer membrane cytochrome OmcA is known to affect attachment to surfaces and partially affect rates of Fe(III)-oxide reduction, this level of restoration from the minimal MtrCAB module was consistent with prior work (Coursolle and Gralnick, 2010 Mol Microbiol 77:995-1008; Myers and Myers, 2001 Applied and Environmental Microbiology 67:260-269; Bretschger et al., 2007 Appl Environ Microbiol 73:7003-7012). Taken together, our data suggests that, in S. oneidensis, MtrC, MtrA, MtrB, and CymA alone are sufficient for reduction of soluble and insoluble Fe(III) compounds by S. oneidensis.

The MtrCAB/CymA Module Confers Fe(III)-Reduction Ability to E. coli.

Having established that the genetic construct could rescue a Shewanella strain lacking key multi-heme cytochromes and structural proteins, we assessed the ability of this module to not only function in E. coli, but function as well as observed in Shewanella.

Culture conditions for E. coli iron reduction experiments were identical to Shewanella except cultures were incubated in anaerobic Balch tubes purged with nitrogen. Reduction activity towards Fe(III) citrate was observed in BL21(DE3) at a rate of 23 μmol Fe(II) L⁻¹day⁻¹. Introduction of pEC86 and pmtrCABcymA resulted in a 4-fold increase in ferric citrate reduction rates (93.7 μmol Fe(II) L⁻¹day⁻¹).

BL21(DE3) also exhibited residual iron reduction activity towards amorphous Fe(III) oxide at a rate of 2.64 μmol Fe(II) L⁻¹day⁻¹. Fe(III) oxide reduction rates increased by 13-fold to 60 μmol Fe(II) L⁻¹day⁻¹ upon incorporation of pEC86 and pmtrCABcymA in BL21(DE3). When 1 μM riboflavin was added to Fe(III) oxide cultures, iron reduction rates increased 7× and 11× for BL21(DE3) and BL21(DE3) pEC86 pmtrCABcymA, respectively.

### The MtrCAB+CymA Module Restores Electricity Production in Shewanella.

In microbial fuel cell systems, one of the main goals is increase current density for maximal power output. Systematically this can be achieved by maximizing surface area, minimizing potential losses, optimizing mass transfer of substrates and redox mediators, and maintaining maximal rates of electron transfer to the anode surface (Wang et al., 2007 Appl. Microbiol. Biotechnol. 76:1439-1446; Logan, 2009 Nat. Rev. Microbiol. 7(5):375-381; Zhou et al., 2011 J. Power Sources. 196:4427-4435). While physical constraints can be optimized through hardware engineering of MFC components, we have presented a microbial engineering approach to create a direct connection to an electrode surface for facile electron transfer.

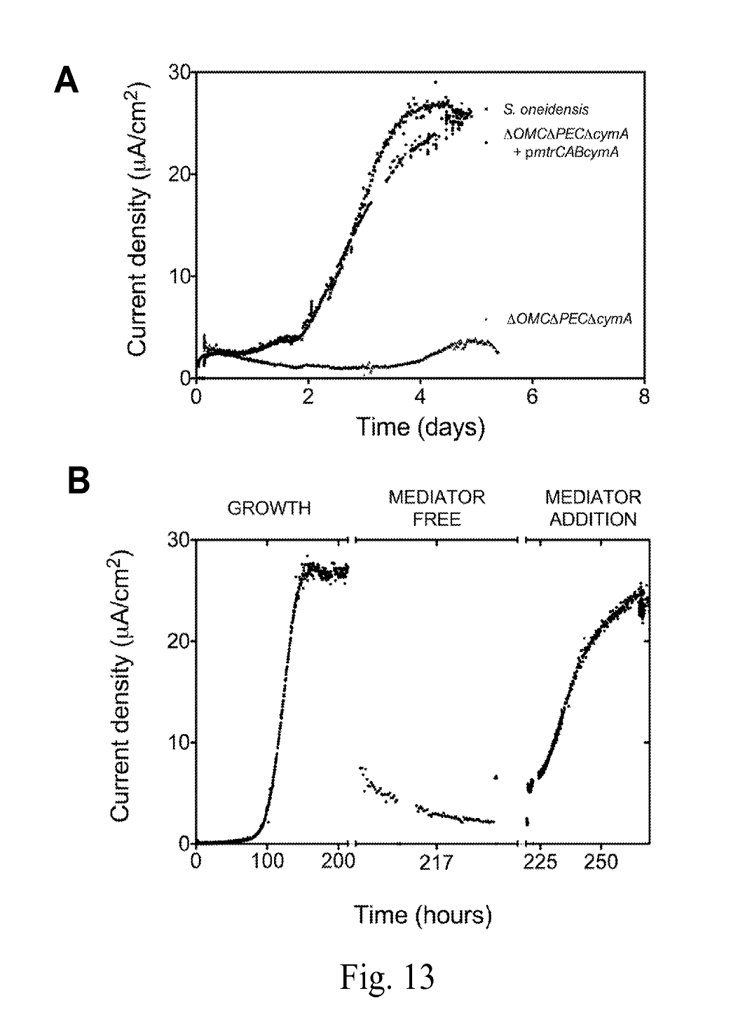

The ability to connect intracellular metabolism to extracellular substrates, especially electrodes, may prove to be a valuable biotechnological tool with many potential applications in bioremediation and production of fuels and chemicals. To demonstrate the requirement of the Mtr pathway for electrode reduction, in particular the four-protein electron conduit of MtrC, MtrA, MtrB and CymA, we examined growth of S. oneidensis, ΔOMC/ΔPEC/ΔcymA and ΔOMC/ΔPEC/ΔcymA pmtrCABcymA in a 3-electrode bioreactor. An anodic (oxidation) current of 2-5 μA was immediately observed upon inoculation of mid- to late-exponential phase for all Shewanella strains tested and WT reached an average plateau current of 26.3 μA/cm²±1.4, ΔOMC/ΔPEC/ΔcymA generated 0.56±0.19 μA/cm² and ΔOMC/ΔPEC/ΔcymA pmtrCABcymA made 21.1±7.9 μA/cm² on an electrode poised at +0.44 V [vs standard hydrogen electrode (SHE)](FIG. 13A). Similar to iron reduction phenotypes, electrode reduction capabilites were fully restored upon expression of the core components of the Mtr respiratory pathway from Shewanella.

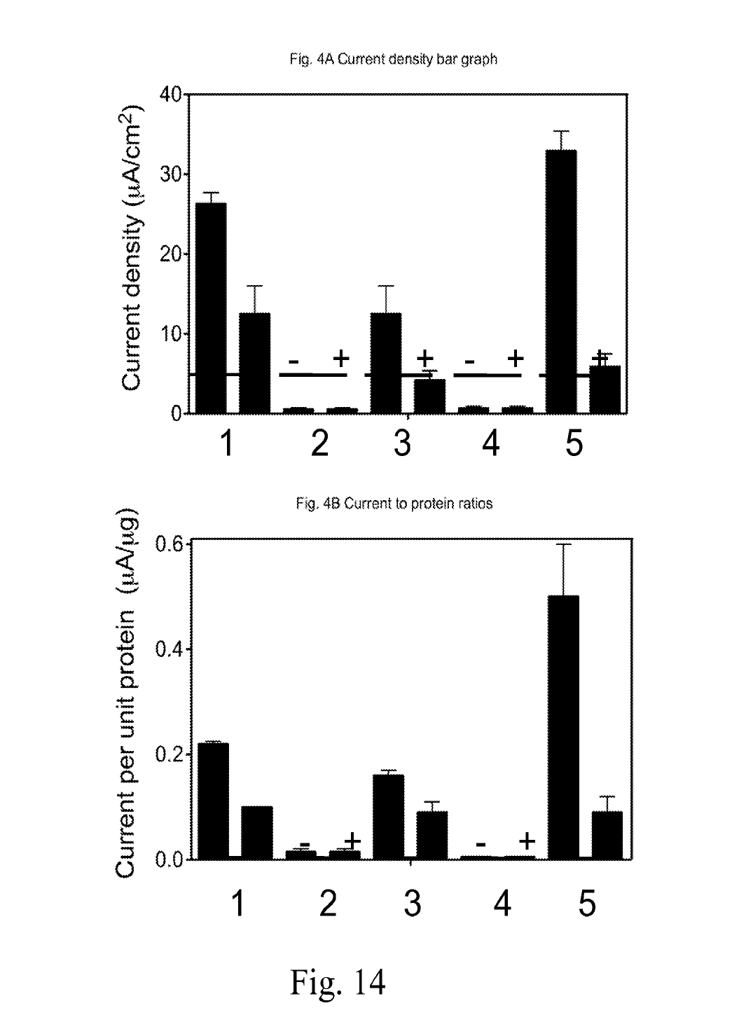

Current densities, while informative from an electrochemical perspective, may not directly resolve the biological significance of electrical output. Therefore, current densities were normalized to total attached protein to determine the specific current of the microbe-electrode interaction and provide a more thorough assessment of current production per cell (i.e. electron transfer efficiency). When normalized for attached biomass, mediator-free S. oneidensis biofilms had an average specific current of 0.1±0.02 μA/μg (FIG. 14B). The WT specific current generated at a carbon electrode poised at +0.44 V vs. SHE was similar to previous findings [0.16 μA/μg; electrode poised at +0.24 V vs SHE (Marsili et al., 2008 Proc Natl Acad Sci USA 105:3968-3973). The specific current decreased to 0.015±0.006 μA/μg (n=3) upon deletion of the Mtr pathway (ΔOMC/ΔPEC/ΔcymA strain). Subsequently, when MtrC, MtrA, MtrB and CymA were overexpressed in the mutant background, a 6-fold increase in specific current [0.09±0.02 μA/μg (n=5) (FIG. 14B; Table 7)] was observed.

The inability of ΔOMC/ΔPEC/ΔcymA to generate current is likely due to a defect in electrode reduction and not in electrode attachment (Table 7). These results show that the defects observed here are likely due to the loss of CymA and other paralogous pathways (MtrDEF) (Coursolle and Gralnick, 2010 Mol Microbiol 77:995-1008). Subsequent disappearance of current upon removal of the Mtr pathway as observed in ΔOMC/ΔPEC/ΔcymA and concurrent recovery of electrode reduction upon expression of MtrC, MtrA, MtrB and CymA in this mutant background, suggests these four proteins are necessary and sufficient for electrode reduction in S. oneidensis.

### MtrCAB+CymA Provides an Electronic Link Between a Non-Electrogenic Bacterium and Electrodes.

Various strains of E. coli have been tested in electrochemical systems and significant current was generated upon addition of exogenous redox mediators (e.g., thionine, neutral red, or ferricyanide) (Emde et al., 1989 Appl. Microbiol. Biotechnol. 32(2):170-175; Park and Zeikus, 1999 J. Bacteriol. 181(8):2403-10; Sakai and Yagishita, 2007 Biotechnol. Bioeng. 98:340-348; Steinbush et al., 2010 Environ. Sci. Technol. 44:513-517). Furthermore, in the absence of added electron shuttles, E. coli has been shown to produce current densities orders of magnitude lower than Shewanella. Under our experimental conditions, without added exogenous redox shuttles, E. coli BL21(DE3) reached an average maximal current density of 0.67±0.24 μA/cm² (FIG. 14A; Table 7). A soluble menaquinone-like mediator with a redox potential centered around 21 mV vs SHE may be responsible for the observed current (Wang et al., 2007 Appl. Microbiol. Biotechnol. 76:1439-1446).

Functional expression of MtrCAB and CymA in E. coli pEC86 resulted in a 49-fold improvement in current density, with a plateau anodic current density of 32.9±2.5 μA/cm² (n=8). Replacement of spent medium (containing planktonic cells and excreted flavins) with fresh mediator-less anaerobic medium resulted in an immediate decrease in average current density [5.9±1.6 μA/cm² (n=8); FIG. 13B; Table 7]. The instantaneous reduction in current after medium replacement in E. coli pEC86 pmtrCABcymA was characteristic of S. oneidensis biofilms (FIG. 14A; Table 7) and suggests that a majority of the observed current is dependent upon a soluble redox mediator generated with our system, possibly secreted by electrode-attached or planktonic cells. When 1 μM of oxidized anaerobic riboflavin was added to a mediator-free biofilm, current slowly increased to a maximal current density that was observed before medium replacement (FIG. 14B). Furthermore, when spent medium containing riboflavin was removed, immediately filtered and added back to the reactor, maximal current densities were obtained and no significant current drop was observed. These results demonstrate that E. coli is capable of direct electrode reduction when properly expressing MtrC, MtrA, MtrB and CymA.

Direct Electrochemical Evidence for Redox Active Components Operating at Similar Potentials in Shewanella and E. coli.

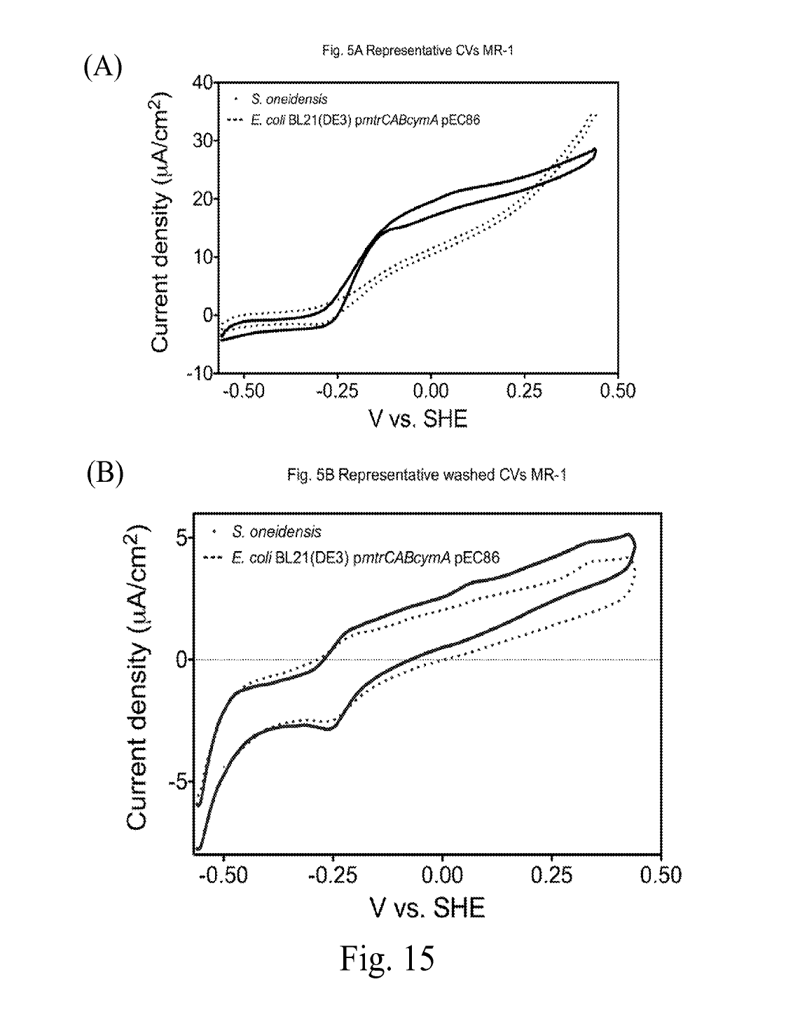

Slow scan rate voltammetry has previously been used to examine electron transfer between electrodes and bacteria (Marsili et al., 2008 Proc Natl Acad Sci USA 105:3968-3973; Srikanth et al., 2008 Biotechnol Bioeng 99:1065-1073; Ross et al., 2011 PLoS ONE 6:e16649). Linear sweeps from a low to high potential and reverse scans back toward a low potental were performed on films of S. oneidensis MR-1, ΔOMC/ΔPEC/ΔcymA, ΔOMC/ΔPEC/ΔcymA pmtrCABcymA, E. coli BL21(DE3) pEC86, and E. coli BL21(DE3) pEC86 pmtrCABcymA, as a current plateau was reached (FIG. 15A), after media exchange (FIG. 15B). After 96 hours of growth at +0.4 V vs SHE, films of S. oneidensis developed a characteristic catalytic wave that began at −0.25 V and rose steeply thereafter with a midpoint potential of −0.2 V, which has been attributed to the presence of flavins (Marsili et al., 2008 Proc Natl Acad Sci USA 105:3968-3973; Baron et al., 2009 J Biol Chem 284:28865-28873). Unlike S. oneidensis, E. coli BL21(DE3) pEC86 pmtrCABcymA exhibited an initial but smaller burst around −0.25 V that leveled off at −0.1 V and at then rose steeply at +0.25 V (FIG. 5A). Upon media exchange, where biofilms were freshly washed but not completely starved of electron donor, as some residual lactate remained, a small catalytic wave centered on −0.2 V, the midpoint potential of flavins, and two smaller peaks at +0.1 V and +0.35 V, were observed for Shewanella and E. coli (FIG. 15B). The similar peak features in washed films suggests a mechanism of reduction by electrode-attached cells driven by the heterologously expressed pathway that operates at the same potentials as the native Mtr pathway.

E. coli Secretes Flavins.

It is well established that S. oneidensis produces and secretes flavins (i.e., flavin adenine dinucleotide (FAD), flavin mononucleotide (FMN), and riboflavin) that enhance the rate of electron transfer to insoluble metals and electrodes (Marsili et al., 2008 Proc Natl Acad Sci USA 105:3968-3973; von Canstein et al., 2008 Appl. Environ. Microbiol. 74(3):615-623; Ross et al., 2009 Appl. Environ. Microbiol. 75(16):5218-5226; Baron et al., 2009 J. Biol. Chem. 284(42):28865-28873; Coursolle et al., 2010 J. Bacteriol. 192(2):467-474). The profile of secreted flavin for anaerobically grown S. oneidensis in defined minimal medium is comprised predominantly of FMN (200-500 nM), and riboflavin (20-100 nM) (von Canstein et al., 2008 Appl. Environ. Microbiol. 74(3):615-623; Covington et al., 2010 Mol. Microbiol. 78:519-532). When cultured aerobically in rich medium, total flavin (FAD, FMN, and riboflavin) can accumulate to concentrations upwards of 1.3 μM (Coursolle et al., 2010 J. Bacteriol. 192(2):467-474). With a direct link between electron transfer rates and flavin concentration (Baron et al., 2009 J. Biol. Chem. 284(42):28865-28873) and the requirement of the Mtr pathway for flavin reduction (Coursolle et al., 2010 J. Bacteriol. 192(2):467-474) we examined the flavin profile of our engineered E. coli strain.

Pushing Electrons into E. Coli: Implications for Electrosynthesis.

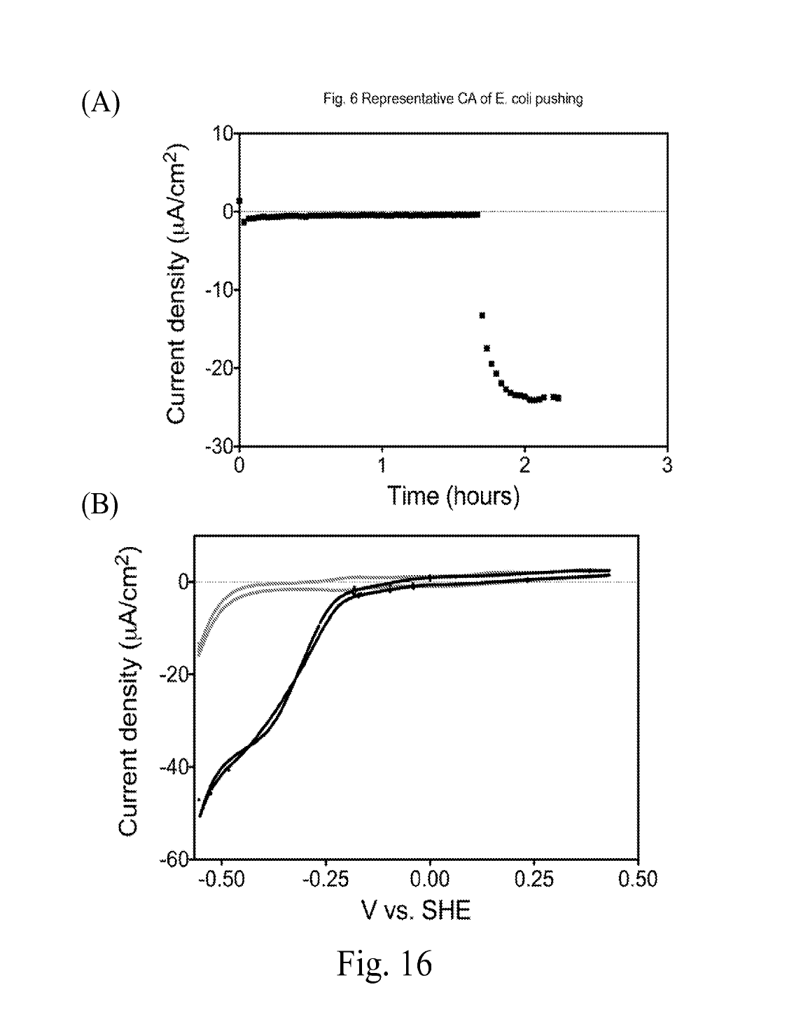

The Mtr pathway is sufficient for electron transfer into S. oneidensis cells attached to a cathode (negatively charged electrode). Once a stable E. coli biofilm was established on a poised electrode (+0.44 V vs SHE), planktonic cells were removed via two media swaps and SBM containing vitamins and minerals was added back. The potential was lowered to −0.36 V vs SHE and after the current stabilized, 50 mM fumarate was added. As has been observed in Shewanella (Ross et al., 2011 PLoS ONE 6:e16649), an immediate decrease in current, i.e. increase in electron flux out of the electrode, was accompanied by the addition of fumarate (FIG. 16A).

Thus, we have demonstrated that electrode-dependent reductive metabolism is possible by modifying an electron flux microbe with one or more modules that include heterologous coding sequences providing non-native metabolic capabilities to the electron flux microbe. Alternatively, electrode-dependent reductive metabolism is possible by modifying a microbe that does not naturally transport electrons across its outer membrane with heterologous coding sequences that confer electron flux functionality.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

TABLE 2
Bacterial strains, vectors, and primers

| Strains | Characteristics and uses | Reference/Source |
|---|---|---|
| S. oneidensis | | |
| MR-1 | Isolated from Lake Oneida, NY | A, B |
| JG612 | Δpta deletion derivative of MR-1 | C |
| E. coli | | |
| K12 | | Lab Stock |
| UQ950 | E. coli DH5a for cloning | D |
| WM3064 | DAP auxotroph donor strain for conjugation | D |
| Vectors | | |
| pBBR1MCS-2 | 5.0 kB broad-host-range-vector for cloning; Km^{r} | E |
| pPET | pBBR1MCS-2 containing pdc and adh (cloned from | This study |
| | Zymomonas moblis, pLOI297) | |
| pGUT2 | pBBR1MCS-2 containing glpD, glpF, glpK, tpiA | This study |
| | (cloned from E. coli K12) | |
| pGUT2PET | pBBR1MCS-2 containing glpD, glpF, glpK, tpiA | This study |
| | (cloned from E. coli K12), pdc and adh (cloned | |
| | from Z. moblis, pLOI297) | |
| Primers | | |
| glpD | | |
| J1 KpnI | GGGGTACCACGAAAGTGAATGAGGGCAGCA | SEQ ID NO: 1 |
| J2 XhoI | CCGCTCGAGCAGGCCAGATTGAAATCTGA | SEQ ID NO: 2 |
| glpFK | | |
| J3 XbaI | GCTCTAGAAGCATGCCTACAAGCATCGTG | SEQ ID NO: 3 |
| J4 NotI | ATAAGAATCGGGCCGCTGCGGCATAAACGCTTCATTCG | SEQ ID NO: 4 |
| tpiA | | |
| J5 SacI | NNGAGCTCCGCTTATAAGCGTGGAGA | SEQ ID NO: 5 |
| J6 SacI | NNGAGCTCGAAAGTAAGTGCCGGATATG | SEQ ID NO: 6 |
| glpABC | | |
| J7 HindIII | CCCAAGCTTGCGCGAAATCAAACAATTCA | SEQ ID NO: 7 |
| J8 EcoRI | CGGAATTCATACATTGGGCACGGAATCG | SEQ ID NO: 8 |
| pUCmod Fwd | | |
| J9 XhoI | NNCTCGAGCCCGACTGGAAAGCGC | SEQ ID NO: 9 |
| pUCmod Rev | | |
| J10 SacI | NNNGAGCTCACATGCGGTGTGAAATACCG | SEQ ID NO: 10 |
| pBBR1MCS-2 Rev | | |
| J11 XhoI | NNNCTCGAGCTCTAGAACTAGTGGATCCC | SEQ ID NO: 11 |

A) Venkateswaran et al., 1999 Int. J Syst. Bacteriol. 49:705-724;
B) Myers and Nealson, 1988 Science 240(4857):1319-1321;
C) Hunt et al., 2010 J. Bacteriol. 192(13):3345-51;
D) Saltikov and Newman, 2003 Proc. Natl. Acad Sci. USA. 100(19):10983-10988;
E) Kovach et al., 1995 Gene 166:175-176.

### Bacterial Strains, Culturing, Growth and Reagents.

S. oneidensis strain MR-1 was previously isolated from Lake Oneida in New York (Myers and Nealson, 1988 Science 240(4857):1319-1321). All strains described in this study can be found in Table 2. Overnight cultures were inoculated from single colonies freshly streaked from a frozen stock into Luria-Bertani (LB) medium (supplemented with 50 μg/mL kanamycin (Km) when required for plasmid maintenance) and incubated for 16 hours. Shewanella Basal Medium (SBM) containing 5 ml/liter of vitamins and trace minerals was used where specified, as described previously (Hau et al., 2008 Appl. Environ. Microbiol. 74(22):6880-6886), and supplemented with 0.05% casamino acids. Anaerobic cultures were placed in Balch anaerobic tubes sealed with butyl rubber stoppers and flushed with nitrogen for 15 minutes (Balch et al., 1979 Microbiol. Rev. 43(2):260-296). All cultures were maintained at 30° C. and shaken continuously at 200 rpm. All molecular biology enzymes were obtained from New England Biolabs (Ipswich, Mass.), TOPO TA cloning vectors were from Invitrogen (Carlsbad, Calif.) and PCR cleanup, gel extraction and plasmid preparation kits were from Qiagen (Valencia, Calif.). All other chemicals were obtained from Sigma (St. Louis, Mo.).

### Plasmid Construction.

Oligonucleotides used are listed in Table 2. To clone glpD, genomic DNA of E. coli K12 was used as a PCR template with primers J1 and J2. PCR products were cloned into pBBR1MCS-2 (Kovach et al., 1995 Gene 166:175-176), creating JF3. To clone glpF and glpK, genomic DNA of E. coli K12 was used as a PCR template with primers J3 and J4 to clone the native glpFK operon. PCR products were cloned into a modified pUC19 previously described (Schmidt-Dannert et al., 2000 Nat. Biotechnol. 18(7):750-753), creating JF4. JF4 was used as a PCR template with primers J9 and J10 designed to incorporate the previously cloned coding sequences in addition to the lac promoter preceding glpF. PCR products were cloned into JF3 creating JF5. To clone tpiA, genomic DNA of K12 was used as a PCR template with primers J5 and J6. PCR products were cloned into JF5 creating pGUT2. Plasmid pLOI297 (Alterthum and Ingram, 1989 Appl. Environ. Microbiol. 55(8):1943-1948) obtained from ATCC (68239), which contains pdc and adhB cloned from Z. mobilis was digested with BamHI and EcoRI. The fragment containing these coding sequences was cloned into pBBR1MCS-2, creating pPET. Plasmid pPET was used as template for a PCR with primer J11 and the standard M13 reverse primer. PCR products A-tailed, then cloned into TOPO TA vector creating JF7. JF7 was digested with XhoI (at a site introduced by the J11 primer) yielding a 3.3 kB band containing a lac promoter, pdc and adhB then cloned into pGUT2 creating pGUT2PET (FIG. 2A). In every case, vector inserts were sequenced to verify accuracy and orientation.

### Growth on Glycerol.

Strains were grown overnight aerobically in LB supplemented with Km (when appropriate), washed twice with SBM and resuspended in SBM. The cells were then inoculated to an optical density at 600 nm (OD₆₀₀) of ˜0.05 into SBM medium containing 50 mM glycerol.

### Resting Cell Assays.

Strains were grown overnight aerobically in LB supplemented with Km, washed twice with SBM and resuspended in SBM. For measuring conversion of lactate to ethanol cells were then inoculated to an optical density at 600 nm (OD₆₀₀) of ˜0.8 into a culture containing 50 mM lactate and 50 mM fumarate and made anaerobic. For measuring conversion of glycerol to ethanol, washed cells were inoculated to an optical density at 600 nm (OD₆₀₀) of ˜0.8 into an anaerobic culture tube containing 40 mM glycerol and 60 mM fumarate. Periodically 0.2 mL aliquots were removed, centrifuged, and supernatants immediately frozen at −80° C. for HPLC analysis.

### HPLC Analysis.

Metabolites were quantified by high performance liquid chromatography (HPLC, all components from Shimadzu Scientific) equipped with UV-Vis detector and refractive index detector. The system consisted of an SCL-10A system controller, LC-10AT Liquid Chromatograph, SIL-10AF autoinjector, RID-10A refractive index detector, SPD-10A UV-Vis detector and CTO-10A column oven. Separation of compounds was performed as described previously (Dharmadi et al., 2006 Biotechnol. Bioeng. 94(5):821-829) with an Aminex HPX-87H guard column and an HPX-87H cation exchange column (Bio-Rad (Hercules, Calif.)). The mobile phase consisted of 0.005N H₂SO₄, set at a flow rate of 0.4 mL/min. The column was maintained at 42° C. and the injection volume was 50 μL.

### Bioreactor Analysis.

Bioreactors were constructed as previously described with modifications (Marsili et al., 2008 Appl. Environ. Microbiol. 74(23):7329-7337). The counter electrode, housed in a glass capillary tube with dialysis tubing at one end, facilitated ion movement but inhibited gas transfer, to avoid any utilization of stray H₂ produced at the counter electrode and allow precise accounting of electron recovery. Isolation of the counter electrode was not necessary for routine glycerol conversion to ethanol. Strains were grown overnight in SBM supplemented with 50 mM glycerol and resuspended in 1 ml of SBM containing 50 mM glycerol and 4 mM riboflavin. The cell suspension was added to 11 ml of the same anaerobic medium in the bioreactor, which was continuously flushed at the counter electrode with nitrogen gas. The electrodes were maintained at an oxidizing potential (+0.44 V vs. SHE) using a 16-channel VMP potentiostat (Bio-Logic SA (Knoxyille, Tenn.)). Current production was monitored over time; 0.2 mL samples were taken periodically for HPLC analysis.

### Enzyme Assays.

Activity assay for glycerol-3-phosphate dehydrogenase was performed as previously described (50). 50 mL cultures of cells to be tested were grown in LB supplemented with 50 mg/mL Km overnight, shaken and incubated (37° C. for E. coli and 30° C. for S. oneidensis). The cultures were centrifuged at 6000×g for 15 minutes and cells were resuspended in 1 mL of 0.1 M sodium phosphate buffered to pH 7.5 (PBS). Cells were then sonicated for 1 minute on ice. Sonicated samples were centrifuged at 15,000×g for 10 min. Glycerol kinase (Hayashi and Lin, 1967 J. Biol. Chem. 242(5):1030-1035), alcohol dehydrogenase (Conway et al., 1987 J. Bacteriol. 169(6):2591-2597) and pyruvate decarboxylase (Ingram et al., 1987 Appl. Environ. Microbiol. 53(10):2420-2425) activity assays in cell lysates were determined as previously described and cell lysates were prepared as described above.

TABLE 3
Total change in substrates and products (in mM). Percentage of carbon
flux going to acetate or ethanol is shown in parenthesis.

| | |
|---|---|
| WT | Δpta |

Fumarate

| | | | | |
|---|---|---|---|---|
| Δglycerol | −19.3 ± 0.6 | | −24.3 ± 1.0 | |
| Δacetate | +4.4 ± 0.2 | (23%) | +2.5 ± 0.1 | (10%) |
| Δethanol | +15.9 ± 1.0 | (82%) | +21.3 ± 0.5 | (88%) |
| Electrode | | | | |
| Δglycerol | −36.1 ± 1.4 | | −32.8 ± 1.5 | |
| Δacetate | +9.1 ± 1.1 | (25%) | +4.9 ± 1.3 | (15%) |
| Δethanol | +26.9 ± 1.6 | (75%) | +27.8 ± 0.5 | (85%) |

### Example 2

### Reagents

Restriction enzymes, phosphatase, DNA polymerase mix, and T4 DNA Ligase were obtained from New England Biolabs (Ipswich, Mass.). TOPO TA cloning kit was obtained from Invitrogen (Carlsbad, Calif.). For PCR cleanup, gel extraction and plasmid preparation, QIAquick PCR Purification Kit, QIAquick Gel Extraction Kit and QIAprep Spin Miniprep Kit from Qiagen (Valencia, Calif.) were used respectively. Sodium fumarate, sodium lactate, and riboflavin were obtained from Sigma (St. Louis, Mo.).

### Bacterial Strains, Plasmids and Growth Conditions.

S. oneidensis was previously isolated from Lake Oneida in New York [1B]. Overnight cultures were inoculated using a single colony from freshly streaked plates in Luria-Bertani (LB) broth. Where noted, Shewanella Basal Medium (SBM) was composed as previously described (Hau et al., 2008 Appl Environ Microbiol 74:6880-6886). Table 4 shows the strains and plasmids used in this study.

TABLE 4
Strains and plasmids used.

| Strain or Plasmid | Characteristics | Reference/Source |
|---|---|---|
| S. oneidensis | Isolated from L. Oneida, NY | A |
| strain MR-1 | | |
| E. coli strain | E. coli DH5α λ(pir) host for | B |
| UQ950 | cloning | |
| E. coli strain | Donor strain (DAP auxotroph) | B |
| WM3064 | for conjugation | |
| JG 686 | S. oneidensis MR-1, ΔfccA | This study |
| JG 1064 | S. oneidensis MR-1, ΔcymA | This study |
| JG 730 | S. oneidensis MR-1, ΔmtrA | C |
| JG 700 | S. oneidensis MR-1, ΔmtrB | C |
| JG 665 | S. oneidensis MR-1, OPEC | D |
| | (periplasmic electron carriers | |
| | ΔmtrA, ΔmtrD, ΔcctA, ΔdmsE, and | |
| | ΔS04360) | |
| JG 300 | menC::mini-Tn10 nptII Kan^{r} | E |
| pSMV3 | 9.1-kb mobilizable suicide vector; | B |
| | oriR6K, mobRP4, sacB, Kan^{r} Ap^{r} | |
| pΔfccA | 2 kb deletion construct for fccA | This study |
| | in pSMV3 | |
| pΔcymA | 2 kb deletion construct for cymA | This study |
| | in pSMV3 | |
| Primers | | |
| fccA UP Fwd | NNACTAGTTGCAGCGGTGCTATTAA | SEQ ID NO: 12 |
| fccA UP Rev | NNGAATTCCATTGCGCCAGAGATCA | SEQ ID NO: 13 |
| fccA DN Fwd | NNGAATTCATCGCGGGTGCATCTGC | SEQ ID NO: 14 |
| fccA DN Rev | NNGAGCTCATGGCAGGCTGATAGGC | SEQ ID NO: 15 |
| cymA UP Fwd | CGGGATCCTGAGCGTTTCAGTGCCTT | SEQ ID NO: 16 |
| cymA UP Rev | CGGAATTCAAATAGTGCACGCCAGTT | SEQ ID NO: 17 |
| cymA DN Fwd | CGGAATTCCCTATCCAAAAGGATAAG | SEQ ID NO: 18 |
| cymA DN Rev | GGACTAGTCCGCATGTTGCCGTTGCA | SEQ ID NO: 19 |

A) Emde et al., 1989 Appl. Microbiol. Biotechnol. 32(2):170-175;
B) Schmidt-Dannert et al., 2000 Nat. Biotechnol. 18(7):750-753;
C) Ross et al., 2007 Appl. Environ. Microbiol. 73:5797-5808;
D) Watson and Logan, 2010 Biotechnol Bioeng. 105(3):489-498;
E) Contiero et al., 2000 J. Ind. Microbiol. Biotechnol. 24(6):421-430.

### Deletion Constructs.

S. oneidensis mutant strains were created as described previously (Hau et al., 2008 Appl Environ Microbiol 74:6880-6886). Briefly, regions upstream and downstream of the gene-of interest were ligated into pSMV3. Subsequent transformation into E. coli WM3064 mating strain, conjugation between the mating strain and S. oneidensis MR-1 and incubation under conditions selecting for removal of the target coding sequence by recombination produced strains with deletions in the desired regions. The menC mutant, previously reported, was generated by transposon insertion using a suicide plasmid with a mini-Tn10 transposon derivative (Newman and Koller, 2000 Nature 405:94-97).

### Electrochemical Techniques.

Bioreactors (electrochemical cells) were prepared as described previously (Marsili et al., 2008 Appl Environ Microbiol 74:7329-7337). The bioreactor consisted of an AXF-5Q graphite (Poco Graphite Company, Decatur, Tex.) working electrode measuring 0.5 cm×2 cm×1 mm, a platinum wire cathode/counter, and a glass frit enclosed, saturated calomel reference electrode connected via a salt bridge (Fisher Scientific, Pittsburgh, Pa.), which was fitted into a Teflon top placed onto a 25 mL glass cone (Bioanalytical Systems, West Lafayette, Ind.). The working electrode was polished with 400-grit sandpaper, rinsed and cleaned in 1 N HCl for 16 hours. It was then attached to a platinum wire using a nylon screw and nut (Small Parts, Inc., Miramar, Fla.). The platinum wire was soldered to an insulated copper wire within a glass capillary tube. The reference electrode salt bridge was maintained with a 5 mm diameter glass capillary tube capped with a nanoporous vycor frit (Bioanalytical Systems, West Lafayette, Ind.) filled with 0.1 M sodium sulfate solution in 1% agarose connected to a larger tube which contained the reference electrode bathed in 0.1 M sodium sulfate. The bioreactors were monitored and potentials were maintained using a 16-channel VMPH potentiostat (Bio-Logic SA, Knoxyille, Tenn.). Anaerobic conditions were maintained with constant flushing of humidified nitrogen gas. The bioreactors were stirred and maintained at 30° C. in a circulating water bath.

### Artificial Biofilm Formation and Characterization.

Thin films of attached cells were prepared as described previously with modifications (Baron et al., 2009 J Biol Chem 284:28865-28873). Overnight cultures (10 mL of >1 O.D. 600) were used to inoculate 400 mL of LB. LB cultures were shaken for 16 hours at 30° C. To facilitate anaerobic culture conditions, cultures were incubated for an additional 5 hours at 30° C. without shaking. Cultures were then centrifuged at 7000×g for 10 minutes. Cell pellets were washed in 25 ml of SBM, centrifuged, and gently resuspended in 10 mL of SBM. The resultant cell suspension was transferred to a sterile, anaerobic 3-electrode bioreactor containing a 2 cm² graphitic working electrode. The working electrode was poised at an oxidizing potential of +0.24 V versus SHE for 16 hours to facilitate attachment of cells to electrodes. The bioreactors were then washed twice with sterile, anaerobic SBM and cyclic voltammetry (CV) was performed (sweeps from −0.56 to +0.44 V versus SHE) to determine baseline features for comparison to subsequent fumarate and flavin additions. The working electrode was poised at a reducing potential of −0.36 V versus SHE and current was monitored until a steady baseline was reached (approximately 1 hour). Fumarate was added to a final concentration of 50 mM and current was monitored.

### Determination of Electrode-Attached Protein.

To quantify attached biomass, electrodes were assayed for total protein as described previously (Coursolle et al., 2010 J Bacteriol 192:467-474; Baron et al., 2009 J Biol Chem 284:28865-28873). Briefly, electrodes were removed from the bioreactor, washed, and incubated in 1 mL of 0.2 N NaOH for 30 minutes at 90° C. to solubilize attached protein. The supernatant was analyzed using the bicinchoninic acid (BCA) assay (Pierce, Rockford, Ill.) according to manufacturer's instructions.

### Example 3

### Bacterial Strains and Plasmids

TABLE 5
Mutant Shewanella and E. coli strains used in this study.

| Strains | Characteristics and uses | Reference/Source |
|---|---|---|
| S. oneidensis | | |
| MR-1 | Isolated from Lake Oneida, NY | A, B |
| JG1854 | MR-1 ΔomcA/ΔmtrC/ΔmtrA/ΔmtrB/ | This work |
| | ΔmtrF/ΔmtrE/ΔmtrD/ΔdmsE/ | |
| | ΔSO4360/ΔcctA/ΔcymA | |
| JG1894 | JG 1854 with pmtrCABcymA | This work |
| JG1825 | MR-1 with pmtrCABcymA | This work |
| E. coli | | |
| JG146 | BL21(DE3) | Lab Stock |
| JG1852 | JG146 with pmtrCABcymA | This work |
| JG1853 | JG146 with pEC86 | This work |
| JG2007 | JG472 with pmtrCABcymA pEC86 | This work |
| UQ950 | E. coli DH5a for cloning | C |
| WM3064 | Donor strain for conjugation, | C |
| | DAP auxotroph | |
| Vectors | | |
| pSMV3 | Deletion vector, Km^{r}, sacB | C |
| pBBR1MCS-2 | 5.0 kB broad-host-range- | D |
| | vector for cloning, Km^{r} | |
| pmtrCABcymA | mtrC and 35 by upstream, mtrA | This work |
| | and 38 by upstream, mtrB and 43 | |
| | by upstream and cymA and x by | |
| | upstream in pBBR-BioBrick | |
| pBBR-BioBrick | pBBRMCS-2 derivative | |
| Complementation | | |
| Primers | 5′ → 3′ | |
| MtrC | NNNAGATCTGTTGGCGCTAGATCATA | SEQ ID NO: 20 |
| | NNNNNNNNNNGCGGCCGCTAATAGG | SEQ ID NO: 21 |
| MtrA | NNAGATCTTTTCTTGAATTTTGTTGG | SEQ ID NO: 22 |
| | NNNNNNNNNNGCGGCCGCGTTGGCT | SEQ ID NO: 23 |
| MtrB | NNNAGATCTCCATCCATCTGGCAAGC | SEQ ID NO: 24 |
| | NNNNNNNNNNGCGGCCGCGGGCTTT | SEQ ID NO: 25 |
| CymA | NNNAGATCTGGAGATAGAGTAATGAA | SEQ ID NO: 26 |
| | NNNNNNNNNNGCGGCCGCCACACTA | SEQ ID NO: 27 |

A) Myers and Nealson, 1988 Science 240(4857):1319-1321;
B) Venkateswaran et al., 1999 Int. J. Syst. Bacteriol. 49:705-724;
C) Saltikov and Newman, 2003 Proc. Natl. Acad. Sci. USA. 100(19):10983-10988;
D) Kovach et al., 1995 Gene 166:175-176.

### Mutant Construction and Complementation.

Mutant strains of S. oneidensis were constructed via targeted coding sequence deletion using homologous recombination (Hau et al., 2008 Appl. Environ. Microbiol. 74(22):6880-6886) and verified by colony PCR using primers flanking the targeted coding sequence as previously described (Coursolle et al., 2010 J. Bacteriol. 192(2):467-474). Using the pBBR/pUC-biobrick system (Coursolle and Gralnick, “Reconstruction of extracellular respiratory pathways for iron(III) reduction in Shewanella oneidensis strain. MR-1,” 2011 Submitted), single and multiple genes were complemented via conjugation (Saltikov and Newman, 2003 Proc Natl Acad Sci USA 100:10983-10988) into S. oneidensis mutant strains. For construction of pmtrCABcymA, each of mtrC, mtrA, mtrB and cymA was amplified from MR-1 genomic DNA using primers 1-8 (Table 5), cloned into the pUC-BioBrick shuttling vector, digested out with XbaI and SpeI, and sequentially ligated into pBBR-biobrick.

### Iron Reduction Assays.

Iron reduction rates were measured for various strains of E. coli and S. oneidensis as described previously (Coursolle et al., 2010 J Bacteriol 192:467-474). Cells were grown aerobically overnight in LB (or LB supplemented with Km when appropriate), washed twice in non-growth SBM, and resuspended in SBM containing vitamins, minerals, and 20 mM lactate. Cells were added to SBM containing 5 mM ferric citrate or amorphous iron oxide to a final O.D.₆₀₀ of 0.13 in a 96-well plate founat within a GasPak System flushed with pure nitrogen for 15 minutes between time points to maintain anaerobicity. Formation of Fe(II) was quantified over time using absorbance of the ferrozine reagent (Stookey, 1970 Analytical Chemistry 42:779-781) at 562 nm measured against a standard of ferrous sulfate dissolved in 0.5 N HCl. Results are shown in Table 6.

TABLE 6
Rates of iron reduction for Shewanella and E. coli

| | nmol/min/mg protein | | |
|---|---|---|---|
| | Fe(III) | Fe(III) | Fe(III) oxide + |
| No added cytochromes | citrate | oxide | 1 μM riboflavin |
| E. coli BL21 (DE3) | 11.2 ± 2.2 | 0.45 ± 0.22 | 5.1 ± 2.0 |
| S. oneidensis MR-1 | 550 ± 12.6 | 22.1 ± 4.7 | 44.4 ± 7.7 |
| S. oneidensis | 18.8 ± 4.3 | 0.58 ± 0.24 | 0.69 ± 0.16 |
| (ΔOMC/ΔPEC/ΔcymA) + | | | |
| pmtrCABcymA | | | |
| E. coli BL21 (DE3) pEC86 | — | — | 43.8 ± 5.9 |
| S. oneidensis | 550 ± 40.4 | 16.9 ± 1.4 | 55.3 ± 5.4 |
| (ΔOMC/ΔPEC/ΔcymA) | | | |

### Electrochemical Techniques and Analysis.

A three-electrode bioreactor system was used for growth on and reduction of a graphite electrode (Marsili et al., 2008 Proc Natl Acad Sci USA 105:3968-3973). From a single colony, cells were grown aerobically overnight in LB (or LB Km for plasmid maintenance when needed). Cultures were centrifuged, washed twice in blank SBM, and resuspended to a final O.D. 600 nm of ˜0.9 in anaerobic SBM containing vitamins, minerals, casamino acids, and 30 mM lactate. Cultures were grown until the current reached a plateau. At this point, cyclic voltammery was performed to obtain catalytic data in the presence of electron donor. Four sweeps from −0.55 V to 0.44 V (vs SHE) were performed to obtain a flavin-less current after two media exchanges (media in the bioreactor chamber was removed and replaced with SBM containing vitamins, minerals, casamino acids, and 30 mM lactate). Once a plateau in current was reached, another set of CVs was performed and chronoamperometry was used to monitor effects of 1 μM riboflavin addition. After addition of flavin, two final CV sweeps were taken and electrodes were washed in SBM and preserved for protein analysis.

### Protein Determination.

Total electrode-associated protein was determined using the BCA Protein Assay Kit (Pierce). Electrodes were removed from the freezer, submerged in 1 mL of 0.2 N NaOH and incubated at 95° C. for 20 minutes. After cooling to room temperature, 25 μL of sample was added to 200 μL of working solution and was incubated at 37° C. for 30 minutes. Absorbance at 562 nm was measured and normalized to protein concentration using a standard curve of bovine serum albumin (0 to 2000 μg/mT).

### Example 4

E. coli strain BL21(DE3) was transformed with pEC86 (cytochrome c maturation genes) and pmtrCABcymA (plasmid containing components of the Mtr respiratory pathway from Shewanella oneidensis MR-1. BL21(DE3) pEC86 pmtrCABcymA was grown at 30° C. overnight in LB supplemented with 50 μg/mL kanamycin and 34 μg/mL chloramphenicol. This cell suspension was centrifuged, washed twice in Shewanella Basal Medium (SBM) and resuspended in anaerobic SBM supplemented with vitamins, minerals, 1 μM riboflavin and 30 mM lactate. Ten milliliters of the final cell suspension (O.D.₆₀₀˜1) was used to inoculate an anaerobic 3-electrode bioreactor with a working electrode poised at +0.44 V vs. SHE. Once a stable plateau current was reached, the medium containing planktonic cells was removed, and replaced with fresh anaerobic SBM (with vitamins, minerals, riboflavin and lactate). After washing, the electrode potential was changed to −0.36 V vs SHE and once a stable current was obtained, anaerobic fumarate was added to a final concentration of 50 mM. Results are shown in FIG. 16.

### Example 5

### Expression of a DHA Module in S. Oneidensis

A plasmid containing glpF and gldA from Escherichia coli was mated into S. oneidensis MR-1. Single colonies were picked from plates and inoculated into 3 mL LB or LB supplemented with 50 μg/mL kanamycin and grown to an OD₆₀₀ of 0.6. This cell suspension was washed twice in SBM and diluted to an OD₆₀₀ of ˜0.05 in SBM containing vitamins, minerals, casamino acids and 50 mM glycerol and grown aerobically. Results are shown in FIG. 17.

### Example 6

Mutants defective in the TCA cycle can be constructed and grown using well-accepted methodology (Coppi et al., 2001 Appl. Eviron. Microbiol. 67:3180-3187; Segura et al., 2008 PLoS Comp. Biol. 4:e360001-360012). Simple insertional deletions are achieved in Geobacter by amplifying ˜500 bp regions up- and downstream of the gene to be deleted, and fused to antibiotic resistance cassettes using complementary primer overhangs. After electroporation of linear fragments into competent cells, selection with the appropriate antibiotic under anaerobic conditions selects clones in which double recombination events have replaced the gene of interest with the antibiotic cassette. For subsequent removal of the gene encoding antibiotic resistance, two alternative protocols may be used. In the first, the linear fragment is constructed with FRT sequences recognized by flp recombinase, and the strain is later transformed with a counterselectable plasmid expressing the recombinase (such as pFLP2). In the second alternative, the original gene replacement is conducted using a fragment of up- and downstream DNA, cloned into a counterselectable plasmid (such as pSMV3) that is inserted into the genome via a single recombination event. These genetic backgrounds can be verified to be defective in TCA cycle function by demonstrating a lack of growth with acetate as the sole carbon an energy source, and growth with hydrogen as an energy source, and supplemental acetate as a carbon source. These mutants can then serve as hosts for recombinant metabolic pathways using either genomic insertions, or transformation with commonly utilized plasmid backbones such as pBBRMCS-1 or pRG5.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

| | |
|---|---|
| SEQ ID | GGGGTACCACGAAAGTGAATGAGGGCAGCA |
| NO: 1 | |
| SEQ ID | CCGCTCGAGCAGGCCAGATTGAAATCTGA |
| NO: 2 | |
| SEQ ID | GCTCTAGAAGCATGCCTACAAGCATCGTG |
| NO: 3 | |
| SEQ ID | ATAAGAATCGGGCCGCTGCGGCATAAACGCTTCATTCG |
| NO: 4 | |
| SEQ ID | NNGAGCTCCGCTTATAAGCGTGGAGA |
| NO: 5 | |
| SEQ ID | NNGAGCTCGAAAGTAAGTGCCGGATATG |
| NO: 6 | |
| SEQ ID | CCCAAGCTTGCGCGAAATCAAACAATTCA |
| NO: 7 | |
| SEQ ID | CGGAATTCATACATTGGGCACGGAATCG |
| NO: 8 | |
| SEQ ID | NNCTCGAGCCCGACTGGAAAGCGC |
| NO: 9 | |
| SEQ ID | NNNGAGCTCACATGCGGTGTGAAATACCG |
| NO: 10 | |
| SEQ ID | NNNCTCGAGCTCTAGAACTAGTGGATCCC |
| NO: 11 | |
| SEQ ID | NNACTAGTTGCAGCGGTGCTATTAA |
| NO: 12 | |
| SEQ ID | NNGAATTCCATTGCGCCAGAGATCA |
| NO: 13 | |
| SEQ ID | NNGAATTCATCGCGGGTGCATCTGC |
| NO: 14 | |
| SEQ ID | NNGAGCTCATGGCAGGCTGATAGGC |
| NO: 15 | |
| SEQ ID | CGGGATCCTGAGCGTTTCAGTGCCTT |
| NO: 16 | |
| SEQ ID | CGGAATTCAAATAGTGCACGCCAGTT |
| NO: 17 | |
| SEQ ID | CGGAATTCCCTATCCAAAAGGATAAG |
| NO: 18 | |
| SEQ ID | GGACTAGTCCGCATGTTGCCGTTGCA |
| NO: 19 | |
| SEQ ID | NNNAGATCTGTTGGCGCTAGATCATA |
| NO: 20 | |
| SEQ ID | NNNNNNNNNNGCGGCCGCTAATAGG |
| NO: 21 | |
| SEQ ID | NNAGATCTTTTCTTGAATTTTGTTGG |
| NO: 22 | |
| SEQ ID | NNNNNNNNNNGCGGCCGCGTTGGCT |
| NO: 23 | |
| SEQ ID | NNNAGATCTCCATCCATCTGGCAAGC |
| NO: 24 | |
| SEQ ID | NNNNNNNNNNGCGGCCGCGGGCTTT |
| NO: 25 | |
| SEQ ID | NNNAGATCTGGAGATAGAGTAATGAA |
| NO: 26 | |
| SEQ ID | NNNNNNNNNNGCGGCCGCCACACTA |
| NO: 27 | |

1. A device comprising:
an electrode; and
at least one microbe in electron communication with the electrode and genetically modified to exhibit increased activity, compared to a wild-type control, of at least one enzyme that catalyzes a metabolic step converting a substrate to a redox-imbalanced pathway product.

2. The device of claim 1 wherein the microbe comprises an electron flux microbe.

3. A device comprising:
an electrode; and
at least one microbe in electron communication with the electrode and genetically modified to exhibit increased activity, compared to a wild-type control, of at least one enzyme that catalyzes electron flux across the microbe's outer membrane.

4. The device of claim 3 wherein the microbe comprises at least one heterologous coding sequence derived from an electron flux microbe.

5. The device of claim 2 wherein the electron flux microbe comprises a member of the genus Geobacter, Pelobacter, Desulfuromonas, Desulfuromusa, Geothermobacter, Geopsycrobacter, Anaeromyxobacter, Desulfovibrio, Desulfobulbus, Geothrix, Clostridium, Deferribacter, Acidomicrobium, Acidithiobacillus, Aeromonas, Bacillus, Desulfitobacterium, Desulfosporosinus, Sporomusa, Rhodoferax, Rhodopseudomonas, Ferrimonas, Ferriglobus, Geoglobus, Gallionella Geothermobacter, Geothermomicrobium, Geovibrio, Pantaea, Pyrobaculum, Thermotoga, Pyrodictium, Sulfobacillus, Sulfospirillum, Shewanella, Sideroxidans, Thermoanaerobacter, Thermococcus, Thermus, Trichlorobacter, Dechloromonas, Azospira, Pseudomonas, Ochrobacterium, Acidiphilum, Therminocola, Vibrio, Marinobacter, Leptothrix, Rhodobacter, Rhodovulum, Chlorobium, Thiodictyon, or Mariprofundus.

6. The device of claim 5 wherein the electron flux microbe comprises a member of the genus Shewanella.

7. The device of claim 1 wherein the substrate comprises a hexose, a pentose, glycerol, a fatty acid, lactate, a mixed hydrocarbon, or an organic acid.

8. The device of claim 1 wherein the product comprises an alcohol, lactate, acetate, succinate, malate, citrate, 1,3-propanediol, ascorbic acid, shikimic acid, 3-hydroxypropanoic acid, dihydroxyacetone, or a biopolymer.

9. The device of claim 8 wherein the biopolymers comprises polyhydroxyalkanoate, polyhydroxybutyrate, or polyhydroxyvalerate.

10. The device of claim 1 wherein the product comprises a fuel.

11. The device of claim 10 wherein the fuel comprises isopropanol, 1-butanol, butanol, 2-methyl-1 butanol, isopentanol, a fatty alcohol, or an olefin.

12. The device of claim 1 wherein the microbe is in physical contact with the electrode.

13. A method comprising:
providing a substrate to a microbe under conditions effective for the microbe to metabolize the substrate to a redox imbalanced product;
wherein at least one microbe is in electron communication with an electrode and metabolic conversion of the substrate to the redox imbalanced product comprises transferring electrons between the electrode and the microbe.

14. A method comprising:
providing a substrate to a microbe under conditions effective for the microbe to metabolize the substrate to a redox imbalanced product;
wherein at least one microbe is in electron communication with an electrode and metabolic conversion of the substrate to the redox imbalanced product exhibits a carbon flux from organic substrate to organic product of at least 80%.

15. The method of claim 13 wherein the electrons are transferred from the microbe to the electrode.

16. The method of claim 13 wherein the electrons are transferred from the electrode to the microbe.

17. The method of claim 13 wherein the microbe comprises an electron flux microbe modified to include at least one heterologous coding sequence that encodes an enzyme that catalyzes a metabolic step of a redox-imbalanced pathway converting a substrate to a product.

18. The method of claim 13 wherein the microbe comprises at least one heterologous coding sequence derived from an electron flux microbe that encodes an enzyme involved in transferring electrons across the microbe's outer membrane.

19. The method of claim 13 wherein the microbe is in physical contact with the electrode.

20. A genetically modified Shewanella oneidensis comprising:
a Shewanella oneidensis microbe genetically modified to exhibit increased activity, compared to a wild-type control, of at least one enzyme that catalyzes a metabolic step converting a substrate to a redox-imbalanced pathway product.

21. A genetically modified Escherichia coli comprising:
an Escherichia coli microbe genetically modified to exhibit increased activity, compared to a wild-type control, of at least one enzyme that catalyzes electron flux across the microbe's outer membrane.

22. The genetically modified Escherichia coli of claim 21 wherein the microbe comprises at least one heterologous coding sequence that encodes at least one of MtrA, MtrB, MtrC.

23. The device of claim 4 wherein the electron flux microbe comprises a member of the genus Geobacter, Pelobacter, Desulfuromonas, Desulfuromusa, Geothermobacter, Geopsycrobacter, Anaeromyxobacter, Desulfovibrio, Desulfobulbus, Geothrix, Clostridium, Deferribacter, Acidomicrobium, Acidithiobacillus, Aeromonas, Bacillus, Desulfitobacterium, Desulfosporosinus, Sporomusa, Rhodoferax, Rhodopseudomonas, Ferrimonas, Ferriglobus, Geoglobus, Gallionella Geothermobacter, Geothermomicrobium, Geovibrio, Pantaea, Pyrobaculum, Thermotoga, Pyrodictium, Sulfobacillus, Sulfospirillum, Shewanella, Sideroxidans, Thermoanaerobacter, Thermococcus, Thermus, Trichlorobacter, Dechloromonas, Azospira, Pseudomonas, Ochrobacterium, Acidiphilum, Therminocola, Vibrio, Marinobacter, Leptothrix, Rhodobacter, Rhodovulum, Chlorobium, Thiodictyon, or Mariprofundus.

24. The device of claim 23 wherein the electron flux microbe comprises a member of the genus Shewanella.

25. The device of claim 23 wherein the substrate comprises a hexose, a pentose, glycerol, a fatty acid, lactate, a mixed hydrocarbon, or an organic acid.

26. The device of claim 23 wherein the product comprises an alcohol, lactate, acetate, succinate, malate, citrate, 1,3-propanediol, ascorbic acid, shikimic acid, 3-hydroxypropanoic acid, dihydroxyacetone, or a biopolymer.

27. The device of claim 26 wherein the biopolymers comprises polyhydroxyalkanoate, polyhydroxybutyrate, or polyhydroxyvalerate.

28. The device of claim 23 wherein the product comprises a fuel.

29. The device of claim 28 wherein the fuel comprises isopropanol, 1-butanol, butanol, 2-methyl-1 butanol, isopentanol, a fatty alcohol, or an olefin.

30. The device of claim 23 wherein the microbe is in physical contact with the electrode.

31. The method of claim 14 wherein the electrons are transferred from the microbe to the electrode.

32. The method of claim 14 wherein the electrons are transferred from the electrode to the microbe.

33. The method of claim 14 wherein the microbe comprises an electron flux microbe modified to include at least one heterologous coding sequence that encodes an enzyme that catalyzes a metabolic step of a redox-imbalanced pathway converting a substrate to a product.

34. The method of claim 14 wherein the microbe comprises at least one heterologous coding sequence derived from an electron flux microbe that encodes an enzyme involved in transferring electrons across the microbe's outer membrane.

35. The method of claim 14 wherein the microbe is in physical contact with the electrode.


SEQUENCE LISTING (27 sequences)

SEQ ID NO: 1 - DNA, 30 bp, artificial
  synthetic oligonucleotide primer
ggggtaccacgaaagtgaatgagggcagca

SEQ ID NO: 2 - DNA, 29 bp, artificial
  synthetic oligonucleotide primer
ccgctcgagcaggccagattgaaatctga

SEQ ID NO: 3 - DNA, 29 bp, artificial
  synthetic oligonucleotide primer
gctctagaagcatgcctacaagcatcgtg

SEQ ID NO: 4 - DNA, 38 bp, artificial
  synthetic oligonucleotide primer
ataagaatcgggccgctgcggcataaacgcttcattcg

SEQ ID NO: 5 - DNA, 26 bp, artificial
  synthetic oligonucleotide primer
nngagctccgcttataagcgtggaga

SEQ ID NO: 6 - DNA, 28 bp, artificial
  synthetic oligonucleotide primer
nngagctcgaaagtaagtgccggatatg

SEQ ID NO: 7 - DNA, 29 bp, artificial
  synthetic oligonucleotide primer
cccaagcttgcgcgaaatcaaacaattca

SEQ ID NO: 8 - DNA, 28 bp, artificial
  synthetic oligonucleotide primer
cggaattcatacattgggcacggaatcg

SEQ ID NO: 9 - DNA, 24 bp, artificial
  synthetic oligonucleotide primer
nnctcgagcccgactggaaagcgc

SEQ ID NO: 10 - DNA, 29 bp, artificial
  synthetic oligonucleotide primer
nnngagctcacatgcggtgtgaaataccg

SEQ ID NO: 11 - DNA, 29 bp, artificial
  synthetic oligonucleotide primer
nnnctcgagctctagaactagtggatccc

SEQ ID NO: 12 - DNA, 25 bp, artificial
  synthetic oligonucleotide primer
nnactagttgcagcggtgctattaa

SEQ ID NO: 13 - DNA, 25 bp, artificial
  synthetic oligonucleotide primer
nngaattccattgcgccagagatca

SEQ ID NO: 14 - DNA, 25 bp, artificial
  synthetic oligonucleotide primer
nngaattcatcgcgggtgcatctgc

SEQ ID NO: 15 - DNA, 25 bp, artificial
  synthetic oligonucleotide primer
nngagctcatggcaggctgataggc

SEQ ID NO: 16 - DNA, 26 bp, artificial
  synthetic oligonucleotide primer
cgggatcctgagcgtttcagtgcctt

SEQ ID NO: 17 - DNA, 26 bp, artificial
  synthetic oligonucleotide primer
cggaattcaaatagtgcacgccagtt

SEQ ID NO: 18 - DNA, 26 bp, artificial
  synthetic oligonucleotide primer
cggaattccctatccaaaaggataag

SEQ ID NO: 19 - DNA, 26 bp, artificial
  synthetic oligonucleotide primer
ggactagtccgcatgttgccgttgca

SEQ ID NO: 20 - DNA, 26 bp, artificial
  synthetic oligonucleotide primer
nnnagatctgttggcgctagatcata

SEQ ID NO: 21 - DNA, 25 bp, artificial
  synthetic oligonucleotide primer
nnnnnnnnnngcggccgctaatagg

SEQ ID NO: 22 - DNA, 26 bp, artificial
  synthetic oligonucleotide primer
nnagatcttttcttgaattttgttgg

SEQ ID NO: 23 - DNA, 25 bp, artificial
  synthetic oligonucleotide primer
nnnnnnnnnngcggccgcgttggct

SEQ ID NO: 24 - DNA, 26 bp, artificial
  synthetic oligonucleotide primer
nnnagatctccatccatctggcaagc

SEQ ID NO: 25 - DNA, 25 bp, artificial
  synthetic oligonucleotide primer
nnnnnnnnnngcggccgcgggcttt

SEQ ID NO: 26 - DNA, 26 bp, artificial
  synthetic oligonucleotide primer
nnnagatctggagatagagtaatgaa

SEQ ID NO: 27 - DNA, 25 bp, artificial
  synthetic oligonucleotide primer
nnnnnnnnnngcggccgccacacta